(19) United States
(12) Patent Application Publication
(10) Pub. No.: US 2012/0021486 A1
(43) Pub. Date: Jan. 26, 2012

(54) ENZYME-BASED NANOSCALE DECONTAMINATING COMPOSITES

(76) Inventors: Cerasela Zoica Dinu, Morgantown, WV (US); Jonathan S. Dordick, Schenectady, NY (US); Ravindra S. Kane, Niskayuna, NY (US); Karl Sanford, Cupertino, CA (US); Gregory M. Whited, Belmont, CA (US); Guangyu Zhu, Troy, NY (US)

(21) Appl. No.: 13/144,530

(22) PCT Filed: Jan. 15, 2010
(86) PCT No.: PCT/US10/00104

§ 371 Date: Sep. 29, 2011

Related U.S. Application Data
(60) Provisional application No. 61/145,415, filed on Jan. 16, 2009.
(60) Provisional application No. 61/205,185, filed on Jan. 16, 2009.

(51) Int. Cl. C12N 9/96; C12N 9/08; C12N 9/14; B82Y 30/00

(52) U.S. Cl. 435/188; 435/195; 435/192; 977/747; 977/742; 977/750; 977/752; 977/902

(57) ABSTRACT

The invention relates to decontaminating composites, and methods, compositions, and kits comprising the same. In some aspects, the invention relates to a decontaminating composite, comprising a perhydrolase associated with a carbon nanotube, that is useful for producing peracids.

### RELATED APPLICATIONS

The present application claims priority under 35 U.S.C. §119(e) to U.S. provisional applications, U.S. Ser. No. 61/145,415, filed Jan. 16, 2009, and U.S. Ser. No. 61/205,185, filed Jan. 16, 2009, both of which are incorporated herein by reference.

### GOVERNMENT SUPPORT

This application was made with U.S. Government support under Grant No. W911SR-05-C-0038, awarded by the U.S. Army Edgewood Chemical Biological Center. The U.S. Government has certain rights in the invention.

### FIELD OF INVENTION

The invention relates to compositions, methods, and kits useful for sterilizing, disinfecting, and cleaning surfaces.

### BACKGROUND

Peracids, such as peracetic acid (PAA), are potent oxidants that exhibit excellent and rapid disinfecting activity against a broad spectrum of pathogens, such as bacteria, yeasts, molds, fungi, spores, viruses, and prions (1-3). As a disinfectant PAA is more effective and is needed at lower concentrations than H₂O₂ (4), while as a sanitizer PAA has been found to be more effective than chlorine to inactivate biofilms on stainless steel surfaces (5). PAA can be used over a wide range of temperatures (0-40° C.) and pHs (3.0-7.5) (6), and it decomposes into nontoxic oxygen, acetic acid, and water. As a result, PAA has been approved by the U.S. Environmental Protection Agency as a pesticide and by the US Food and Drug Administration for direct food contact and food contact surfaces. PAA has also been used to disinfect medical supplies (7, 8), and increasingly used for waste water treatment and pulp and textile bleaching (9). Enzymatic methods for producing peracids have been developed. However, the effectiveness of peracid producing enzymes as practical disinfectant reagents have not been fully realized.

Commercial PAA is generally produced by reacting acetic acid with H₂O₂ using sulfuric acid as the catalyst. However, this reaction is typically slow (requiring up to several days to yield high amounts of PAA), and moreover, residual levels of acetic acid, H₂O₂, and corrosive sulfuric acid in the product are typically high (10). As an alternative to chemical synthesis, several biocatalytic routes have been devised, which involve hydrolases, e.g., lipases, esterases, and cholinesterases. These enzymes catalyze perhydrolysis of acyl donor substrates in the presence of H₂O₂ to generate peracids under mild reaction conditions (11-13). Lipases, in particular, are well known to generate peracids in nonaqueous media, which are then able to oxidize alkenes stoichiometrically to generate epoxides and peracids (14-16). For example, Novozyme 435 (immobilized lipase B from Candida antarctica on acrylic resin), was used to generate peracids either by direct synthesis from carboxylic acids and H₂O₂ or by perhydrolysis of carboxylic acid esters (17). The enzymatically produced PAA was found to have sporicidal activity similar to that of commercial PAA. However, the major drawback of using hydrolases is their very low perhydrolytic activity in aqueous solutions and fast deactivation by high concentrations of H₂O₂ and the resulting PAA.

### SUMMARY OF THE INVENTION

Functional composites have been developed that have enzymatic activities useful for a range of applications, including, but not limited to, disinfecting, sanitizing, bleaching and cleaning. In particular, it has been discovered that enzymes, e.g., hydrolases, perhydrolases, haloperoxidases, which are capable of generating oxygen metabolites, e.g., superoxides, peroxides, peracids, oxidized halides, can be associated with materials to produce such composites. The composites retain sufficient enzyme activity compared with that of free-enzyme to be effective at producing oxygen metabolites at concentrations sufficient to kill or inactivate a variety of different infectious agents. The composites also have improved transport properties, and thus, can be effectively combined with other materials, e.g., polymers to produce decontaminating compositions that can be used repeatedly without a substantial loss in enzyme activity. In particular, decontaminating compositions, such as polymeric and paint compositions, that are useful for coating surfaces can be produced.

In some aspects, composites are provided that comprise a material associated with a peracid-producing enzyme, e.g., a perhydrolase. In some embodiments, composites are provided that comprise a material associated with a haloperoxidase. Typically, the material is a nanomaterial, such as a silica-based nanomaterial or a carbon nanotube, e.g., a single-walled or multi-walled carbon nanotube. The material may be of a variety of sizes or shapes and may be functionalized to achieve certain desired properties, e.g., aqueous solubility. The enzyme may be associated with the material covalently or non-covalently. Linkers, such as polyethylene glycol, may be used to bridge the association between a enzyme and a material. Such linkers can improve the enzyme activity of the composite by avoiding reductions in enzyme activity that can result from direct enzyme attachment. Methods of producing the composites are also provided.

In some aspects, decontaminating compositions comprising any of the composites disclosed herein are provided. The decontaminating compositions are often useful for coating a surface. Thus, typically the decontaminating compositions comprise a composite and a material, e.g., a polymer, e.g., a film-forming polymer, that renders the composition suitable for coating a surface. In some embodiments, the decontaminating compositions comprise a composite and poly(methyl methacrylate) polymer or poly(vinyl acetate) polymer. In other embodiments, paint compositions are provided that comprise a composite and a polymer suitable for use in a paint, e.g., a latex-based paint. Such paint compositions may also include binders, pigments, and other additives commonly found in paints. Typically, decontaminating compositions comprise an effective amount of the composite. Methods of producing the decontaminating compositions are also provided.

In other aspects, methods of decontaminating a surface are provided. The methods typically comprise coating the surface with any one of the decontaminating compositions disclosed herein. Oxygen metabolites, e.g., peracids, that kill or inactivate infectious agents may be produced on the coated surface by contacting the surface with an appropriate enzyme substrate, e.g., an acetate ester, e.g., propylene glycol diacetate (PGD) and, typically, also contacting the surface with a peroxide, e.g., H₂O₂.

Methods of coating a surface with a decontaminating composition are also provided. The methods typically involve steps for coating a surface with a decontaminating composition that comprises an effective amount of any of the composites disclosed herein. The methods may involve depositing a film of the decontaminating composition onto the surface using chemical solution deposition, chemical vapor deposition or physical vapor deposition, for example. In other embodiments, the surface is spin-coated with, or dipped in, a decontaminating composition. In another embodiment, the decontaminating composition is painted onto the surface. Surfaces are typically coated with film have a thickness in a range of about 200 nm to about 2 mm, and a variety of surfaces may be coated. For example the surface to be coated may be a plastic, a metal, a wood, a paper, a ceramic, a composite, a polymeric, or a textile surface.

In another aspect, decontaminating kits are provided. The kits may comprise, for example, a container housing a composite and instructions for using the composite to produce a decontaminating composition. In some embodiments, the kits comprise a container housing the decontaminating composition and instructions for coating a surface with the decontaminating composition. Other components may also be included in the kits, including, but not limited to, a container housing a reagent for determining perhydrolase activity, a container housing a reagent for decontaminating the surface, and instructions for decontaminating a surface.

### DEFINITIONS

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

As used herein, the term “acetate ester” refers to an ester of acetic acid, with the general formula CH₃CO₂R, where R is an organic substituent group, having one free valence at a carbon atom, e.g. CH₃CH₂—, ClCH₂—, CH₃C(═O)—.

As used herein, the term “activating” refers to modifying a molecule, to facilitate a subsequent reaction involving the molecule. For example, activating a carbon nanotube may comprise conjugating amine-reactive sulfo-NHS esters onto the carbon nanotube to facilitate a subsequent reaction in which another molecule, e.g., PEG, perhydrolase, is covalently associated with the carbon nanotube.

As used herein, the term “acyl donor substrate” is a molecule from which an acyl group may be derived. An “acyl group” is a functional group derived by the removal of one or more hydroxyl groups from an oxygen containing acid, usually a carboxylic acid. An acyl group has the formula RCO, where R represents an alkyl group that is attached to the CO group with a single bond. In some embodiments, an acyl group is derived from an acyl donor substrate when combined with perhydrolase in the presence of a peroxide under conditions that facilitate perhydrolysis.

As used herein, the phrase “associated with” or “associating with” refers to the state of molecular entities being coupled together or the process of coupling molecular entities together, respectively. Molecular entities may be associated with each other by covalent or noncovalent interactions.

As used herein, the term “bleaching” refers to the elimination of colors, typically by oxidation. Bleaching may be complete or partial.

As used herein, the term “carbon nanotube” refers to an allotrope of carbon having a cylindrical nano structure.

As used herein, the term “cleaning” refers to the removal of undesired substances, contaminants, infectious agents, or microbes from a surface. Cleaning may be complete or partial.

As used herein, the term “composite” refers to a substance that comprises two or more different molecular entities. A composite may also be referred to, in some instances, as a “conjugate”.

As used herein the term “decontaminating” refers to killing, eliminating, inhibiting or inactivating infectious agents, e.g., microbes, viruses, prions, and/or chemical agents, e.g., nerve agents or biological warfare agents. Decontaminating may be complete or partial. Similarly, the term “decontaminant” refers to a substance capable of decontaminating a surface. In some embodiments, a decontaminant may inactivate, inhibit, eliminate, or kill at least 20%, at least 30%, at least 40%, at least 50%, at least 60%, at least 70%, at least 80%, at least 90%, at least 95%, or at least 99% of the infectious agents or chemical agents on a surface.

As used herein, the term “decontaminating composite” refers to a substance that comprises two or more different molecular entities that directly or indirectly elicits a desired effect on an infectious agent and/or a chemical agent. In some embodiments, a decontaminating composite comprises a material, e.g., particle, nanotube, associated with an enzyme, e.g., a hydrolases, a perhydrolase, haloperoxidase, capable of generating a product, e.g., an oxidizing agent, e.g., a peracid, at sufficient levels for killing or inhibiting the growth of microbes, particularly pathogenic microbes, and/or for eliminating or inactivating other infectious agents, e.g., viruses, prions, and/or chemical agents, e.g., nerve agents or biological warfare agents.

As used herein, the term “disinfecting” refers to killing, eliminating, inhibiting, e.g., inhibiting the growth of, or inactivating infectious agents, e.g., microbes, viruses, prions. Disinfecting may be complete or partial. Similarly, the term “disinfectant” refers to a substance capable of disinfecting a surface. It is not intended that the embodiments disclosed herein be limited to any particular surface or infectious agents. In some embodiments, a disinfectant may inactivate or kill at least 20%, at least 30%, at least 40%, at least 50%, at least 60%, at least 70%, at least 80%, at least 90%, at least 95%, or at least 99% of the infectious agents on a surface.

As used herein, the term “disinfecting composite” refers to a substance that comprises two or more different molecular entities that directly or indirectly elicits a desired effect on an organism, particularly a microorganism or other infectious agent. In some embodiments, a disinfecting composite comprises a material, e.g., particle, nanotube, associated with an enzyme, e.g., a hydrolases, a perhydrolase, haloperoxidase, capable of generating a product, e.g., an oxidizing agent, e.g., a peracid, at sufficient levels for killing or inhibiting the growth of microbes, particularly pathogenic microbes, or for eliminating or inactivating other infectious agents, e.g., viruses, prions.

As used herein, the phrase “effective amount” refers to a quantity of an agent, e.g., a disinfecting composite, a decontaminating composite, that is necessary to achieve the activity required of the agent for a specific application, e.g., disinfecting, decontaminating. Such effective amounts are readily ascertained by one of ordinary skill in the art and are based on many factors, such as the nature of the composition comprising the composite.

As used herein, the term “film” refers to a thin layer of a material. In some embodiments, a film has a thickness in a range of about 1 nm to about 200 nm. In some embodiments, a film has a thickness in a range of about 200 nm to about 2 μm. In some embodiments, a film has a thickness in a range of about 2 μm to about 200 μm. In some embodiments, a film has a thickness in a range of about 200 μm to about 2 mm. A film may be transparent, translucent or opaque.

As used herein, the term “film-forming polymer” refers to a polymer capable of forming a film.

As used herein, the term “functionalizing” refers to modifying a molecule to confer a desired functionality to the molecule. For example, functionalizing a carbon nanotube may comprise modifying the carbon nanotube to produced free carboxyl groups, e.g., by treatment with an acid, to improve solubility of the nanotube in an aqueous solution.

As used herein, the term “GDSL motif” refers to an active site motif comprising an amino acid sequence of GDSL, or a functional variant thereof.

As used herein, the term “haloperoxidase” refers to a peroxidase that mediates the oxidation of halides, or organic halogen compounds, in the presence of peroxides, e.g., hydrogen peroxide.

As used herein, the term “infectious agent” refers to an agent capable of producing an infectious disease or illness in a subject including, but not limited to, pathogenic microbes, viruses, bacteria, fungi, protozoa, multicellular parasites, and prions.

As used herein, the term “linker” refers to a molecular entity suitable for forming a connecting structure between at least two other molecular entities. Typically, the linker is a flexible molecule, e.g., a polyethylene glycol polymer. Also, the linker is typically inert.

As used herein, the term “micromaterial” refers to a material, e.g., a particle, having at least one dimension, e.g., a diameter, in a range of 1 μm to less than about 1 mm.

As used herein, the term “nanomaterial” refers to a material, e.g., a particle, a nanotube, having at least one dimension, e.g., a diameter, in a range of 1 nm to less than 1 μm.

As used herein, a coding sequence and regulatory sequences are said to be “operably joined” or “operably linked” when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein.

As used herein, the term “perhydrolase” refers to a molecule, e.g., an enzyme, a protein, a polypeptide, that is capable of catalyzing a reaction that results in the formation of sufficiently high amounts of peracid suitable for a desired application such as, for example, cleaning, bleaching, decontaminating and disinfecting. In some embodiments, perhydrolases disclosed herein produce high perhydrolysis to hydrolysis ratios (e.g., greater than 1). The high perhydrolysis to hydrolysis ratios of these enzymes make them suitable for use in a wide variety of applications. In some embodiments, the perhydrolase is a M. smegmatis perhydrolase or variant or homolog thereof.

As used herein, the phrase “perhydrolysis to hydrolysis ratio” refers to a relationship between the amount of an acid produced by perhydrolysis to the amount of the acid produced by hydrolysis under defined conditions and within a defined time.

As used herein, the term “perhydrolysis” refers to a reaction of a substrate with peroxide to form a peracid.

As used herein, the term “sanitizing” refers to reducing the number of microorganisms or infectious agents to a safe level.

As used herein, a “vector” may be any of a number of nucleic acid molecules into which a nucleic acid having a desired sequence may be inserted by restriction and ligation for transport between different genetic environments or for expression in a host cell. Vectors are typically composed of DNA although RNA vectors are also available. Vectors include, but are not limited to, plasmids, phagemids, and virus genomes. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art, e.g., galactosidase or alkaline phosphatase, and genes which visibly affect the phenotype of transformed or transfected cells, hosts, colonies or plaques, e.g., green fluorescent protein.

### BRIEF DESCRIPTION OF DRAWINGS

FIG. 1 shows perhydrolase S54V (denoted as AcT) catalyzed perhydrolysis of PGD to generate peracetic acid (PAA).

FIG. 2 shows covalent attachment of AcT onto multi-walled carbon nanotubes (MWNTs). FIG. 2a shows direct attachment of AcT onto MWNT. In addition to covalent binding, nonspecific hydrophobic interactions also exists due to the large size and hydrophobic nature of AcT. The insert shows a TEM image of AcT-MWNT conjugates. FIG. 2b shows attachment of AcT onto MWNT using discrete PEG (dPEG) spacers.

FIG. 3 shows a structure of AcT. FIG. 3a shows AcT octamer with catalytic triad Ser11, Asp 192, and His195 shown in filled space, and all other residues shown with lines; colored residues, green: hydrophobic, pale blue: hydrophilic, dark blue: basic, and red: acidic. (b) Molecular surface of monomeric AcT; colored residues, green: hydrophobic, pink: hydrogen bonding, and blue: mild polar.

FIG. 4a shows specific activity of AcT-nanotube conjugates compared to free AcT. FIG. 4b kinetics of free AcT and AcT-dPEG-NT conjugates.

FIG. 5 shows specific activity of the composites relative to that of ACT-dPEG-MWNT conjugates. Black bars indicated thick films (200 μm for polymeric composites and 400 μm for paint composite) and grey bars indicated thin films (ca. 2 μm for polymeric composites).

FIG. 6 shows stability of the paint composite tested under different conditions: () dry state at room temperature; (▾) immersed in water at room temperature; (▪) dry state at 50° C.; and (♦) immersed in water at 50° C.

FIG. 7 shows PAA generated in 20 min by paint composite at different loadings of AcT-dPEG-MWNT conjugates. The paint has a surface area of 5 cm² and thickness of 400 μm. Reactions were conducted in 1 ml potassium phosphate buffer (50 mM, pH7.1) containing 100 mM H₂O₂ and 100 mM PGD.

FIG. 8a shows the amino acid composition of AcT. High aliphathy (non-aromatic hydrophobics) residues in the AcT protein structure. The protein has an aliphatic index of 95.66 and a grand average of hydropathicity (GRAVY) of 0.117 based on biocomputation. FIG. 8b shows a structural depiction of the AcT enzyme complex. The catalytic Triad is composed of Ser11, Asp192, and His19 shown in space fill, with all other residues shown with lines. Coloring: green=hydrophobic, pale blue=hydrophilic, dark blue=basic, and red=acidic. Each chain has distinct color. Dotted lines indicate hydrogen bonding or VDW contacts. Inspection reveals only one hydrogen bond and one VDW contact holding two adjoining monomers together on one side. Hydrophobic intercalation is responsible for the remaining binding interactions, largely via one tryptophan on one monomer and one phenylalanine on the other monomer interacting with smaller hydrophobics on the surface.

FIG. 9 shows a TEM image of AcT-PEG-MWNTs. As shown, the surface of the nanotube is covered by protrusions, which are attributed to the AcT attachment.

FIG. 10 shows Attenuated Total Reflection Fourier Infrared Spectroscopy (ATR-FTIR) of PEG-functionalized MWNTs and AcT-PEG-MWNTs respectively. The amide bond formation is visible at 1650 cm⁻¹ with a 50% increase when the AcT protein was attached to the MWNTs.

FIG. 11 shows a comparison between the thickness of spin-coated films versus the spin coater speed.

FIG. 12A shows a reaction scheme for conjugating an enzyme to a multi-walled carbon nanotube. FIG. 12B shows a scheme for coating a surface with a composition comprising a perhydrolase-nanotube composite. FIG. 12C shows a scheme for performing a perhydrolase activity assay on a surface coated with a composition comprising a perhydrolase-nanotube composite.

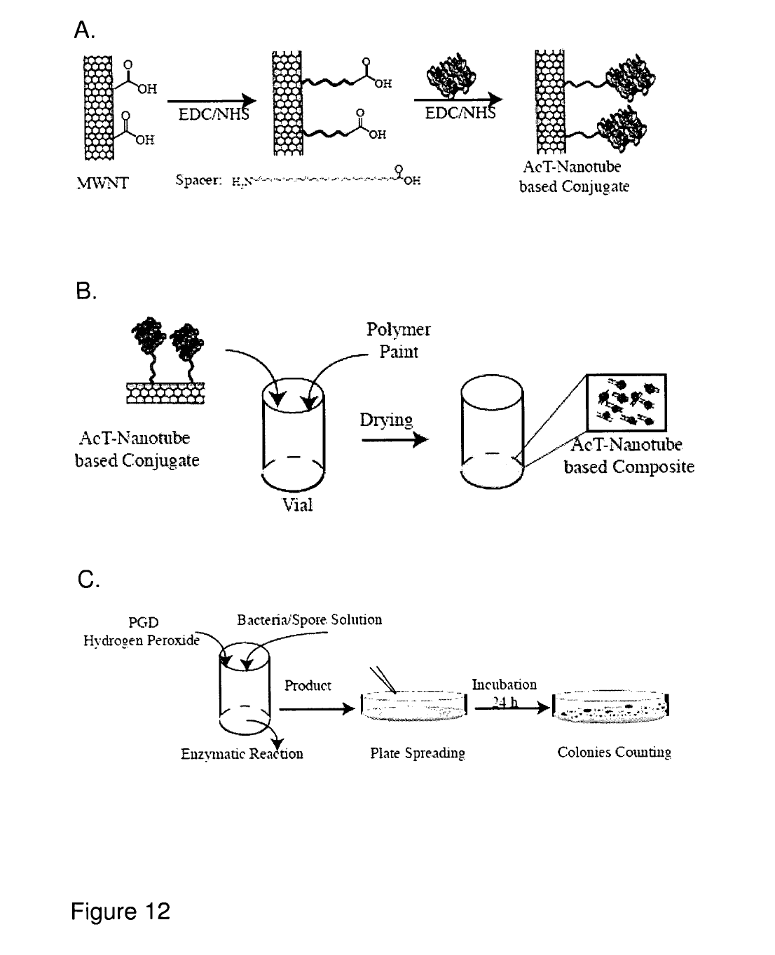

FIG. 13 shows an analysis of latex paint and PVAc based compositions. FIGS. 13A-C show scanning electron microscopic analyses of latex paint films comprising perhydrolase composites. FIG. 13D shows a surface characterization of a PVAc based film comprising perhydrolase composites.

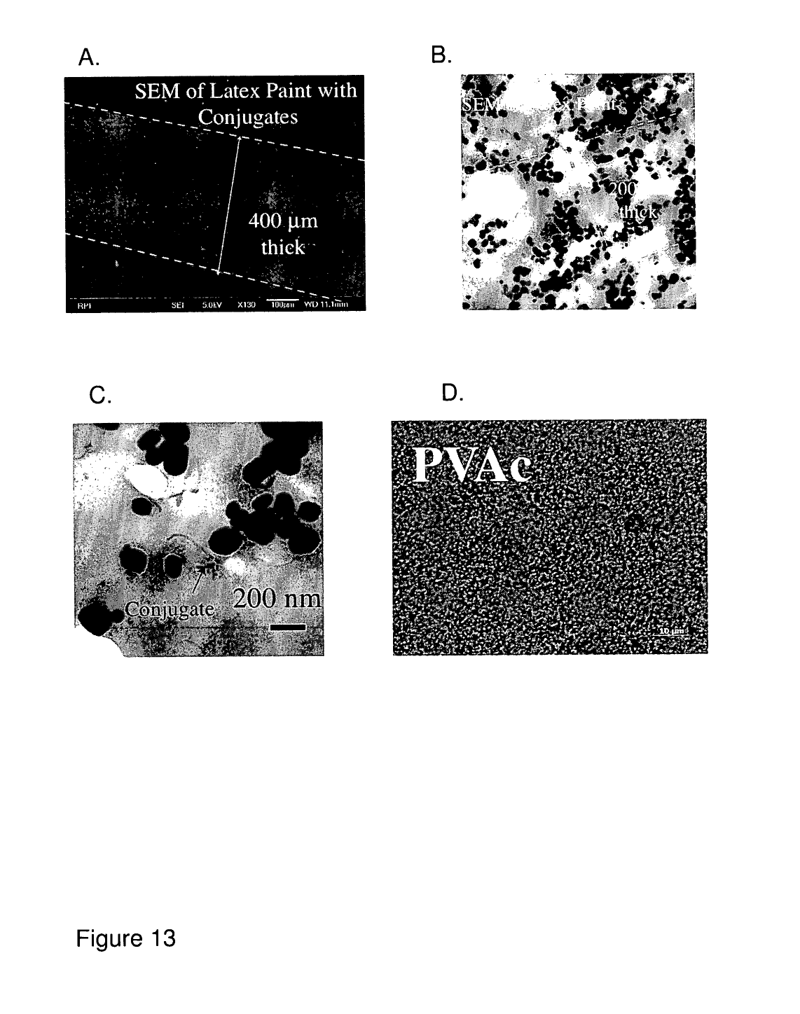

FIG. 14 shows high purity B. cereus spores by Transmission Electron Microscopy (TEM) (FIG. 14A) and light microscopy (FIG. 14B).

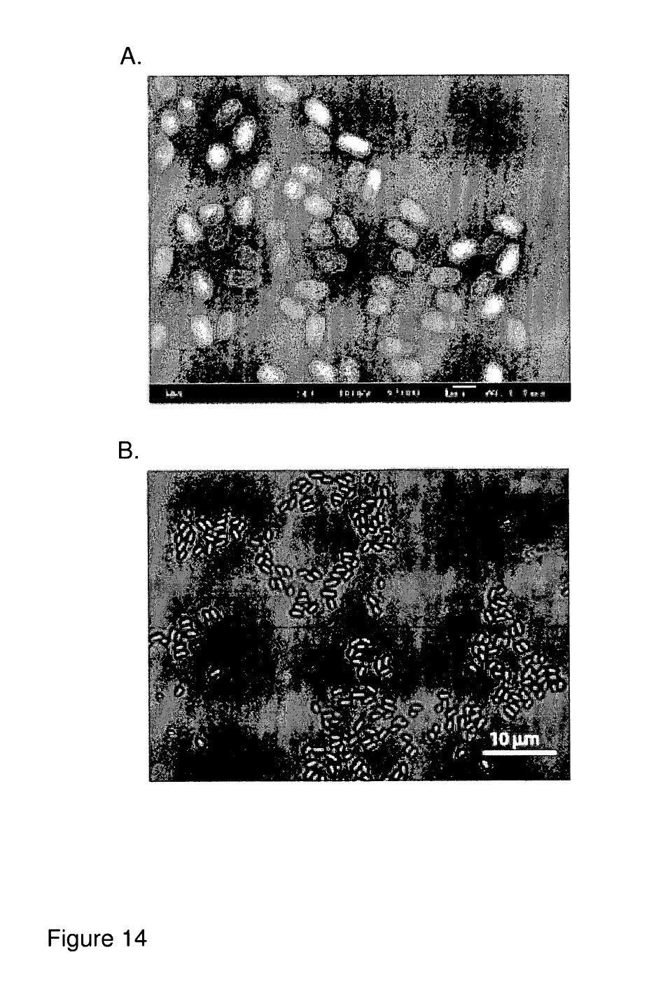

FIG. 15 shows sporicidal activity of paint compositions. Tests were performed against 10⁶ CFU/ml of B. cereus spores. FIGS. 15A and B show PAA generated with 100 nM Hydrogen Peroxide at various concentrations of conjugates in paint ranging from 0.008 wt % to 0.08% wt and at various reaction cycle numbers ranging from 1 to 5. FIG. 15C shows sporicidal effects of the paint composites at different reaction times (10 and 30 minutes) and different H₂O₂ concentrations (10 mM, 25 mM, 50 mM, and 100 mM). FIG. 15D shows sporicidal effects of PAA in 50 mM Hydrogen Peroxide at different reaction times (10, 30, and 60 minutes) and different conjugate concentrations.

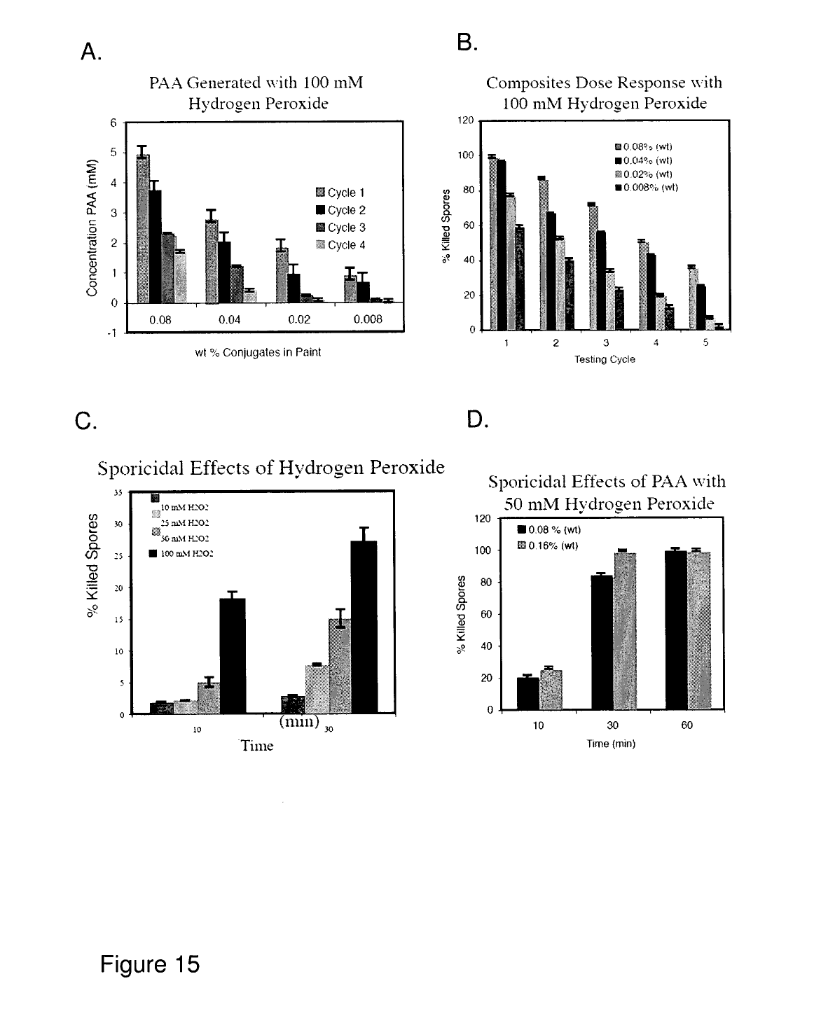

### DETAILED DESCRIPTION OF CERTAIN EMBODIMENTS OF THE INVENTION

Disclosed herein are composites having enzymatic activities useful for a variety of different applications, such as decontaminating, disinfecting, bleaching, and cleaning. The composites typically comprise a material associated with at least one enzyme capable of catalyzing a reaction that yields a product useful for a decontaminating, disinfecting, bleaching, sterilizing, or cleaning. Frequently, the composites comprise a material associated with a perhydrolase. In some cases, the composites comprise a material associated with a haloperoxidase. A perhydrolase is a molecule, typically a protein or polypeptide, that is capable of catalyzing a reaction that results in the formation of a peracid (also referred to as a peroxyacid). Peracids are powerful oxidizing agents, and thus, are useful for a variety of applications, including decontaminating, disinfecting, bleaching or cleaning applications.

### Composites

A variety of different enzymes, e.g., perhydrolases, haloperoxidases, may be used in the composite disclosed herein. A perhydrolase, for example, is a protein that effectively catalyzes the perhydrolysis of an acetate ester, such as propylene glycol diacetate (PGD), to generate a peracid, such as peracetic acid (PAA). PAA is a potent oxidant increasingly used for sanitization, decontamination, disinfection, and sterilization due to its broad effectiveness against bacteria, yeasts, molds, fungi, and spores. In the instant disclosure, examples of decontaminating compositions that can generate sufficiently high amount of PAA, suitable for sporicidal activity, are developed by incorporating perhydrolase-carbon nanotube composites into compositions comprising polymers, including coatings such as paint. Compositions comprising these composites are active and stable, with no detectable composites leaching after repeated use.

Structural and functional data of perhydrolases are available in the art, for example, as disclosed in U.S. Patent Application Publication Number US2007/0167344. Typically, perhydrolases comprise a GDSL active site motif (Akoh C. C., et al., GDSL family of serine esterases/lipases. Progress in Lipid Research 43 (2004) 534-552). Examples of proteins comprising a GDSL active site motif including, but are not limited to, proteins assigned to the following NCBI Reference Sequence numbers: CAK07736.1, EEG54823.1, EEG25819.1, ABC90722.1, AAD02335.1, ACS56084.1, ACP25067.1, ACM36508.1, ACM26483.1, ACJ52052.1, ACI54896.1, ABR69058.1, ABR59931.1, ABO01318.1, ABM58078.1, ABL94451.1, ABK70783.1, ABI46443.1, ABG63022.1, ABG11277.1, AAK89941.1, AAK87224.1, EEZ86767.1, AAK65750.2, ABB08071.1, AAZ61308.1, AAK65755.1, EEW36308.1, EEW30249.1, EEW30244.1, CAD18396.1, EEV01101.1, EET62845.1, EES81694.1, CAQ59034.1, EER67650.1, EAS50304.1, EEQ60873.1, EEQ59360.1, BAB47978.1, EEP23692.1, BAB16197.1, BAB16192.1, ABW33605.1, ABW33600.1, CAK23508.1, EEJ51115.1, EEG95280.1, EEG94478.1, EEG88510.1, EEG36514.1, EEE46462.1, CAQ37111.1, EDY31802.1, EDY18942.1, CAI89235.1, ACE90994.1, CAC46027.1, EDS89554.1, EAL45518.1, EDT39558.1, EDS21743.1, EDS20259.1, EDR99390.1, EDP13950.1, EDO057532.1, EDQ02700.1, EDQ32781.1, CAJ93221.1, EBA40639.1, EBA11941.1, EBA09025.1, EBA01361.1, EAW28065.1, EAV42665.1, EAV41708.1, EAV41703.1, EAU52929.1, EAU42453.1, CAD73431.1, EAR61662.1, EAR52921.1, EAR28559.1, EAR18590.1, EAQ66348.1, EAQ65062.1, EAP70910.1, CAC14575.1, BAA97789.1, BAA97784.1, YP_{—}726589.1, NP_{—}436338.2, NP_{—}436343.1, YP_{—}469449.1, YP_{—}368715.1, YP_{—}730806.1, YP_{—}674187.1, YP_{—}296152.1, NP_{—}865746.1, NP_{—}385554.1, NP_{—}354439.1, NP_{—}102192.1, NP_{—}066659.1, NP_{—}066654.1, YP_{—}997096.1, YP_{—}341681.1, YP_{—}890535.1, YP_{—}767838.1, NP_{—}522806.1, YP_{—}941241.1, YP_{—}642333.1, NP_{—}357156.1, XP_{—}650904.1, ZP_{—}03711894.1, ZP_{—}06176963.1, ZP_{—}06113219.1, ZP_{—}05813433.1, ZP_{—}05813428.1, ZP_{—}05807753.1, ZP_{—}04743741.1, ZP_{—}04668295.1, ZP_{—}04667652.1, ZP_{—}01549793.1, ZP_{—}01549788.1, ZP_{—}01548748.1, ZP_{—}01227536.1, ZP_{—}01166094.1, ZP_{—}05344422.1, ZP_{—}05115863.1, ZP_{—}01446757.1, ZP_{—}04835067.1, ZP_{—}04776942.1, ZP_{—}04448835.1, ZP_{—}03991690.1, ZP_{—}03800931.1, ZP_{—}03759083.1, ZP_{—}03753325.1, ZP_{—}03752480.1, ZP_{—}03716554.1, ZP_{—}03168756.1, ZP_{—}03130435.1, ZP_{—}02929364.1, ZP_{—}02909299.1, ZP_{—}02440209.1, ZP_{—}02439242.1, ZP_{—}02417667.1, ZP_{—}02382879.1, ZP_{—}02167177.1, ZP_{—}02155699.1, ZP_{—}02088279.1, ZP_{—}02075177.1, ZP_{—}01771332.1, ZP_{—}01750267.1, ZP_{—}01745666.1, ZP_{—}01735405.1, ZP_{—}01612648.1, ZP_{—}01437456.1, ZP_{—}01154811.1, ZP_{—}01134367.1, ZP_{—}01123955.1, ZP_{—}01076843.1, ZP_{—}01075501.1, ZP_{—}00946593.1, YP_{—}002975623.1, YP_{—}002257153.1, YP_{—}002255320.1, YP_{—}002281122.1, YP_{—}001326766.1, YP_{—}001073808.1, YP_{—}001961023.1, YP_{—}001961018.1, YP_{—}001224805.1, YP_{—}001338993.1, YP_{—}002322430.1, YP_{—}001978172.1, YP_{—}002825820.1, YP_{—}002549516.1, YP_{—}002544410.1, XP_{—}001914102.1, XP_{—}001913897.1, and XP_{—}001913669.1.

The perhydrolase used in the composites may be a member of the SGNH hydrolase super-family, which is assigned NCBI accession number cl01053, or the SGNH hydrolase family, which is assigned NCBI accession number cd00229. SGNH hydrolases (also referred to as GDSL hydrolases) are a group of related lipases and esterases that have an active site that closely resembles the typical Ser-His-Asp(Glu) triad from other serine hydrolases, but may lack the carboxlic acid (Mølgaard A., et al., Rhamnogalacturonan acetylesterase elucidates the structure and function of a new family of hydrolases. Structure 2000, 8:373-383; Wei Y., et al. A novel variant of the catalytic triad in the Streptomyces scabies esterase. Nat Struct Biol. 1995 March; 2(3):218-23.). The tertiary fold of SGNH hydrolases is also substantially different from that of the alpha/beta hydrolase family and unique among all known hydrolases. The perhydrolase may also be a member of the SGNH arylesterase like subfamily, which is assigned NCBI accession number cd01839. Perhydrolases of the SGNH hydrolase subfamily are similar to arylesterase (7-aminocephalosporanic acid-deacetylating enzyme) of A. tumefaciens. More than one perhydrolase may be used.

An example perhydrolase that is useful in the composites has the following amino acid sequence:

(SEQ ID NO: 1;
NCBI Reference Sequence number YP_890535)
MAKRILCFGDSLTWGWVPVEDGAPTERFAPDVRWTGVLAQQLGADFEVI
EEGLSARTTNIDDPTDPRLNGASYLPSCLATHLPLDLVIIMLGTNDTKA
YFRRTPLDIALGMSVLVTQVLTSAGGVGTTYPAPKVLVVSPPPLAPMPH
PWFQLIFEGGEQKTTELARVYSALASFMKVPFFDAGSVISTDGVDGIHF
TEANNRDLGVALAEQVRSLL.

This perhydrolase is derived from Mycobacterium smegmatis and is a member of the SGNH hydrolase super-family, the SGNH hydrolase family, and the SGNH arylesterase like subfamily.

Another example of a perhydrolase is a variant of the perhydrolase derived from Mycobacterium smegmatis that has a valine substituted for the serine at amino acid position 54. The amino acid sequence of this variant is set forth as:

(SEQ ID NO: 3)
MAKRILCFGDSLTWGWVPVEDGAPTERFAPDVRWTGVLAQQLGADFEVI
EEGLVARTTNIDDPTDPRLNGASYLPSCLATHLPLDLVIIMLGTNDTKA
YFRRTPLDIALGMSVLVTQVLTSAGGVGTTYPAPKVLVVSPPPLAPMPH
PWFQLIFEGGEQKTTELARVYSALASFMKVPFFDAGSVISTDGVDGIHF
TEANNRDLGVALAEQVRSLL.

In some instances, this perhydrolase may be referred to herein as “perhydrolase S54V” or “AcT”.

In some cases, the perhydrolase may be selected from among those disclosed in U.S. Patent Application Publication Numbers US2009/0311198, US2008/0145353, US2007/0244021, US2007/0167344 and US2005/0281773, the relevant contents of which are incorporated herein by reference.

Haloperoxidases may also be used with the composites disclosed herein. Haloperoxidases are peroxidases that mediate the oxidation of halides, or pseudohalides, by hydrogen peroxide. Examples of haloperoxidases include, but are not limited to, chloroperoxidase, bromoperoxidase, and iodoperoxidase. Mammalian haloperoxidases, such as myeloperoxidase, lactoperoxidase and eosoniphil peroxidase, which are capable of oxidizing the pseudohalide thiocyanate (SCN-), may also be used. Haloperoxidases may be obtained from any biological source or may be synthesized. Haloperoxidases are also readily obtained from commercial sources, such as Novozymes, Biodesign, International, Sigma, and DMV International. More than one haloperoxidase may be used. Methods for evaluating haloperoxidase activity are disclosed US2009/0104172, the contents of which are incorporated herein by reference.

Haloperoxidases are members of the Peroxidase, family 2 which is assigned NCBI Accession: cl03166. Chloroperoxidase is a versatile heme-containing enzyme that exhibits peroxidase, catalase and cytochrome P450-like activities in addition to catalyzing halogenation reactions. Chloroperoxidase has been isolated from Caldariomyces fumago (Conesa A, et al., J. Bio. Chem. Vol. 276, No. 21, Issue of May 25, pp. 17635-17640, 2001). An example chloroperoxidase that is useful in the composites has the following amino acid sequence: MFSKVLPFVGAVAALPHSVRQEPGSGIGYPYDNNTLPYVAPGPTDSRAPCPALNALA NHGYIPHDGRAISRETLQNAFLNHMGIANSVIELALTNAFVVCEYVTGSDCGDSLVN LTLLAEPHAFEHDHSFSRKDYKQGVANSNDFIDNRNFDAETFQTSLDVVAGKTHFDY ADMNEIRLQRESLSNELDFPGWFTESKPIQNVESGFIFALVSDFNLPDNDENPLVRID WWKYWFTNESFPYHLGWHPPSPAREIEFVTSASSAVLAASVTSTPSSLPSGAIGPGAE AVPLSFASTMTPFLLATNAPYYAQDPTLGPNDKREAAPAATTSMAVFKNPYLEAIGT QDIKNQQAYVSSKAAAMASAMAANKARNL (SEQ ID NO: 2; NCBI Reference Sequence number P04963). Examples of haloperoxidases also include enzymes of the group EC 1.11.1.10, according to the enzyme nomenclature set forth by the International Union of Biochemistry and Molecular Biology.

Functional homologs or variants of the enzymes disclosed herein may also be used in the composites. In general, homologs typically will share at least 60% homology, at least 70% homology, at least 80% homology, at least 85% homology, at least 90% homology, at least 95% homology, at least 97% homology, at least 98% homology, at least 99% homology, or least 99.5% homology with a enzyme and retain sufficient enzyme activity for a desired application. The homology can be determined using methods well known in the art. Variants will typically share at least 80% amino acid identity, at least 85% amino acid identity, at least 90% amino acid identity, at least 95% amino acid identity, at least 97% amino acid identity, at least 98% amino acid identity, at least 99% amino acid identity, or least 99.5% amino acid identity with a enzyme and retain sufficient enzyme activity for a desired application. For example, homology or amino acid sequence identity can be determined using various, publicly available software tools including, but not limited to, FASTA @ EBI, BLAST, and CLUSTAL.

Assays for perhydrolase activity are disclosed herein and are well known in the art. Examples of assays for perhydrolase activity are found in U.S. Pat. No. 7,510,859, U.S. Pat. No. 7,384,787, US2009/0311198, US2008/0145353, US2007/0244021, and US2005/0281773, the contents of which are incorporated herein by reference. A typical perhydrolase-activity assay comprises contacting a composite with a peroxide, e.g., H₂O₂ and an acyl donor substrate and measuring the level of peracid produced. The level of peracid produced can be determined directly or indirectly, e.g., by evaluating antimicrobial activity.

The enzymes, e.g., perhydrolases, disclosed herein may be produced by any of a variety of methods known in the art. For example, the perhydrolases may be chemically synthesized, may be produced by recombinant methods, or may be purified from natural sources. In some aspects, expression vectors comprising an isolated nucleic acid molecule encoding a perhydrolase are provided herein. Typically an expression vector comprises a coding sequence of a perhydrolase (protein coding sequence or functional RNA sequence) operably linked to a promoter. Host cells transformed or transfected with such expression vectors also are provided.

A variety of different materials may be used in the composites disclosed herein. The material may be of any suitable size, shape, or composition, depending on the application. Although in some cases the material may be a micromaterial, typically the material is a nanomaterial. The material may be cylindrical, spherical, or polyhedric, for example. The material may comprise a glass and/or a polymer such as polyethylene, polystyrene, silicone, polyfluoroethylene, polyacrylic acid, a polyamide (e.g., nylon), polycarbonate, polysulfone, polyurethane, polybutadiene, polybutylene, polyethersulfone, polyetherimide, polyphenylene oxide, polymethylpentene, polyvinylchloride, polyvinylidene chloride, polyphthalamide, polyphenylene sulfide, polyester, polyetheretherketone, polyimide, polymethylmethacylate and/or polypropylene. In some cases, the material may be silica-based or comprise a ceramic such as tricalcium phosphate, hydroxyapatite, fluorapatite, aluminum oxide, or zirconium oxide. However, typically the material is a carbon nanotube, which may be single-walled or multi-walled, or a related carbon structure, such as, for example, a nanotorus, nanobud, cup-stacked nanotube or fullerene structure.

The carbon nanotubes used in the composites disclosed herein may be of a variety of sizes and/or aspect ratios. For example, the carbon nanotubes may have an outer diameter in a range of about 1 nm to about 10 nm, about 10 nm to about 20 nm, about 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, or about 90 nm to about 100 nm. The carbon nanotubes may have an outer diameter of about 0.3 nm, about 0.4 nm, about 0.5 nm, about 0.6 nm, about 0.7 nm, about 0.8 nm, about 0.9 nm, about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm or more. The carbon nanotubes may a length in a range of about 0.05 μm to about 0.1 μm, of about 0.1 μm to about 0.5 μm, about 0.5 μm to about 1 μm, about 1 μm to about 2 μm, about 1 μm to about 5 μm, about 5 μm to about 10 μm, about 5 μm to about 20 μm, about 10 μm to about 20 μm, about 20 μm to about 50 μm, about 50 μm to about 100 μm, about 100 μm to about 200 μm, about 200 μm to about 500 μm, about 500 μm to about 1 mm, or about 1 mm to about 20 mm. The carbon nanotubes may have a length of about 0.1 μm, about 0.5 μm, about 1 μm, about 2 μm, about 5 μm, about 10 μm, about 20 μm, about 50 μm, about 100 μm, about 200 μm, about 500 μm, about 1 mm, or more. The carbon nanotubes may have a length-to-diameter ratio in a range of 10¹:1 to 10²:1, 10²:1 to 10³:1, 10³:1 to 10⁴:1, 10⁴:1 to 10⁵:1, 10⁵:1 to 10⁶:1, or 10⁶:1 to 10⁷:1, for example.

Carbon nanotubes may be produced by any appropriate method known in the art. A variety of art-known methods have been developed to produce nanotubes including, but not limited to, arc discharge, laser ablation, high pressure carbon monoxide (HiPCO), and chemical vapor deposition (CVD). Examples of methods for producing carbon nanotubes are disclosed in U.S. Pat. No. 7,622,314; U.S. Pat. No. 7,566,478; U.S. Pat. No. 7,553,472; U.S. Pat. No. 6,203,814; U.S. Pat. No. 6,303,094; and U.S. Pat. No. 6,325,909 the contents of which are incorporated herein by reference. Carbon nanotubes may also be obtained from any of a variety of commercial sources (e.g., NanoIntegris in Skokie, Ill., MkNano in Mississauga, Ontario or Nanolabs in Newton, Mass.).

Frequently, the materials of the composites are modified to confer a desired functionality to the material. Such materials may be referred to herein as functionalized materials, e.g., functionalized carbon nanotubes. For example, a carbon nanotube may be modified to possess free carboxyl groups, e.g., by treatment with an acid, to improve solubility of the nanotube in an aqueous solution and/or facilitate a subsequent reaction involving the nanotube. For example, a carbon nanotube may be treated with a mixture of sulfuric acid and nitric acid to functionalize the surface of the nanotube. The acid treatment typically results in a large concentration of carboxylic acid groups on the nanotube surface, and may also generate other groups (e.g., —OH), depending on the reaction conditions. Other acids that may be used for functionalizing carbon nanotubes include, but are not limited to: hydrochloric acid, phosphoric acid, boric acid, hydrofluoric acid, hydrobromic acid, acetic acid, ascorbic acid, butanoic acid, carbonic acid, chromic acid, citric acid, formic acid, heptanoic acid, hexanoic acid, hydrocyanic acid, lactic acid, nitrous acid, octanoic acid, oxalic acid, pentanoic acid, propanoic acid, sulfurous acid, and uric acid.

Amide (NH₂) functionalized nanotubes may also be prepared, e.g., as a derivative of carboxyl functionalized nanotubes. The carboxyl group may be reacted with SOCl₂ to form an acyl chloride, which is then reacted with dimethylamine to produce amide functionalized nanotubes. Other appropriate modifications will be apparent to the skilled artisan.

Functionalized carbon nanotubes may also be obtained directly from a commercial source. For example, NanoLab, Inc. offers functionalized nanotubes having free carboxyl or amide groups.

Functionalized carbon nanotubes, e.g., having free carboxyl groups, may have altered solubility properties, e.g., improved aqueous solubility. The functionalized carbon nanotubes may be soluble in an aqueous solution (at a pH of about 6.5 to about 7.5) at a concentration in a range of about 0.01 mg/ml to about 0.05 mg/ml, about 0.05 mg/ml to about 0.1 mg/ml, about 0.1 mg/ml to about 0.5 mg/ml, about 0.5 mg/ml to about 1.0 mg/ml, about 1 mg/ml to about 5 mg/ml, about 5 mg/ml to about 10 mg/ml, about 10 mg/ml to about 50 mg/ml, about 50 mg/ml to about 100 mg/ml. The functionalized carbon nanotubes may be soluble in an aqueous solution (at a pH of about 6.5 to about 7.5) at a concentration of about 0.01 mg/ml, about 0.05 mg/ml, about 0.1 mg/ml, about 0.5 mg/ml, about 1 mg/ml, about 5 mg/ml, about 10 mg/ml, about 50 mg/ml, about 100 mg/ml, or more.

A material of a composite may be covalently or noncovalently associated with a perhydrolase. Examples of noncovalent interactions between materials and perhydrolases include, but are not limited to, hydrogen bonds, ionic bonds, van der Waals forces, hydrophobic interactions, and combinations thereof. In some cases, the perhydrolase is associated with the material by adsorbtion.

When enzymes are covalently associated with a material, a linker molecule is sometimes used between the enzyme and material. Linkers encompass any moiety that is useful to connect an enzyme with a nanomaterial. Any of a variety of linker molecules can be used. Typically, the linker is flexible and avoids reductions in perhydrolase activity that are observed when a perhydrolase is directly associated with a material. Linkers may be polymers comprising nucleic acids, amino acids, carbohydrates, or ethers, for example. Linkers may be hydrocarbons chains. The linker may be a glycine-rich or alanine-rich peptide. It is frequently desirable to use linkers that reduce nonspecific interactions with a material, that do not decrease the solubility of the material, and that enhance perhydrolase activity, e.g., by improving the surface hydrophilicity of the material. The linker may comprise polyethylene glycol (PEG), polypropylene glycol (PPG), or combinations thereof. PEG linkers of a variety of length may be used depending on the application and desired perhydrolase activity. Linkers may have a length in a range of about 1 monomer to about 10 monomers, about 10 monomers to about 20 monomers, about 20 monomers to about 50 monomers, about 50 monomers to about 100 monomers, about 100 monomers to about 200 monomers, about 200 monomers to about 500 monomers, about 500 monomers to about 1000 monomers, or about 1000 monomers to about 2000 monomers. PEG linkers may have a length of about 1 monomer, about 10 monomers, about 20 monomers, about 50 monomers, about 100 monomers, about 200 monomers, about 500 monomers, about 1000 monomers, or about 2000 monomers.

The activity of the composites disclosed herein may be tuned in a variety of ways. For example, the activity may be tuned by associating a material with a perhydrolase having a known activity level, by controlling the number of perhydrolases associated with each material, and by associating a material with combinations of different perhydrolases.

By selecting a perhydrolase with a high-level of activity (compared with a reference value) for association with a material, a composite having a proportionally high-level of activity can be produced. Alternatively, by selecting a perhydrolase with a low-level of activity (compared with a reference value) for association with a material, a composite having a proportionally low-level of activity can be produced. Similarly, by selecting a combination of perhydrolases having high- and low-levels of activity for association with a material, a composite having an intermediate level of activity can be produced. Perhydrolases having different levels of activity are known in the art and can be selected as described herein to produce composites having a spectrum of different activity levels. U.S. Patent Application Publication Number US2008/0145353, for example, discloses a variety of perhydrolase variants that exhibit higher- or lower-levels of activity compared with a wild-type perhydrolase derived from Mycobacterium smegmatis. By evaluating the activity of composites having different perhydrolases the skilled artisan can readily select the appropriate perhydrolase, or combination of different perhydrolases, in order to achieve a desired activity level.

The activity of a composite can also be tuned by increasing the number of perhydrolase molecules per material. Typically, the activity of the composite increases in a direct relationship with increases in the ratio of perhydrolase molecules to material. Accordingly, by evaluating the activity of composites at various perhydrolase-to-material ratios the skilled artisan can readily select the appropriate ratio in order to achieve a desired activity level. For example, the number of perhydrolase molecules per material, e.g., nanomaterial, may be in a range of about 1 to about 50, about 50 to about 100, about 100 to about 200, about 200 to about 300, about 300 to about 400, about 400 to about 500, about 500 to about 1000, about 1000 to about 2000, about 2000 to about 5000, or about 5000 to about 10,000. The number of perhydrolase molecules per material, e.g., nanomaterial, is about 50, about 100, about 200, about 300, about 400, about 500, about 1000, about 2000, about 5000, about 10,000, or more.

Other parameters that may be adjusted to tune the activity of the composite will be apparent to the skilled artisan. Non-limiting examples of other parameters include the nature of the interaction(s) mediating the association of the material with the perhydrolase (covalent vs. non-covalent, use of a linker), type of linker, length of linker, etc. In each case, by evaluating the activity of composites under different conditions (e.g., non-covalent vs. covalent), the skilled artisan can readily select the appropriate conditions to achieve a desired activity level in a composite.

An enzyme, e.g., perhydrolase, that is covalently or non-convalently associated with a material, e.g., nanomaterial, may maintain an activity level that is at least 5%, at least 10%, at least 20%, at least 30%, at least 40%, at least 50%, at least 60%, at least 70%, at least 80%, at least 90%, at least 95%, or at least 99% of the activity level of the free (unbound) enzyme.

Enzymes may be covalently linked with materials using any of a variety of art known methods. For example, when the material is a carbon nanotube, a perhydrolase may be covalently associated with the nanotube by activating the nanotube and combining the activated carbon nanotube with the perhydrolase under conditions that facilitate covalent attachment of the perhydrolase with the activated carbon nanotube. Thus, a typical reaction involves mixing a first solution comprising an activated carbon nanotube with a second solution comprising the perhydrolase (or a combination of different perhydrolases) under conditions that result in covalent attachment of the perhydrolase with the nanotube. The relative concentrations of the perhydrolase and activated nanotubes in the first and second solution may be adjusted to achieve a desired perhydrolase-to-material ratio in the composite.

When a linker is to be used, the activated carbon nanotube is typically first combined with the linker under conditions that result in covalent attachment of the linker to the nanotube. Subsequently, the linker is activated and the carbon nanotube comprising the activated linker is combined with the perhydrolase under conditions that result in covalent attachment of the perhydrolase to the linker. In a typically reaction, a first solution comprising the activated carbon nanotube is mixed with a second solution comprising the linker under conditions that result in covalent attachment of the linker with the carbon nanotube. The carbon nanotube-linker conjugate is then activated and a third solution comprising the activated carbon nanotube-linker conjugate is mixed with a fourth solution comprising the perhydrolase under conditions that result in covalent attachment of the perhydrolase with the carbon nanotube-linker conjugate.

A variety of covalent and non-covalent linking chemistries may be used for attaching enzymes to materials, including, but not limited to, any of a variety of click chemistries. See, for example, the methods disclosed in Jennifer L. Brennan, et al., Bioconjugate Chem. 2006, 17, 1373-1375; M-E Aubin-Tam et al Biomed Mater. 2008 September; 3(3):034001; and Click Chemistry for Biotechnology and Materials Science, Edited by Joerg Lahann Wiley (Dec. 21, 2009). Activation often involves the formation of amides, esters, ethers, C—C bonds, or S—S bonds. Standard EDC-NHS activation reactions may be used, e.g., for activating nanotubes and linkers. Methods and associated reagents for carrying out EDC-NHS reactions are available from commercial sources, e.g., Thermo Scientific, Waltham, Mass. Typically, in the EDC-NHS reaction, 1-Ethyl-3-[3-dimethylaminopropyl]carbodiimide hydrochloride (EDC) and N-hydroxysulfosuccinimide (NHS) are combined with the molecule to be activated, e.g., the carbon nanotube or linker molecule, under conditions that generate amine reactive Sulfo-NHS-esters on the molecule.

As will be appreciated by the skilled artisan, the methods disclosed herein for covalently associating perhydrolases with materials are not intended to be limiting and other methods known in the art may be appropriate, particularly when materials other than nanotubes are used, including, for example, silica-based, ceramic, and polymeric materials.

### Decontaminating Compositions and Methods

The composites typically comprise a perhydrolase that is capable of catalyzing a reaction that results in the formation of peracids, which are powerful oxidizing agents that are useful for a variety of applications. Accordingly, decontaminating compositions comprising composites are also provided herein. In some embodiments, disinfecting compositions are provided. Typically, the compositions comprise a polymer, or combination of polymers, and a composite. However, the compositions are not limited to those comprising polymers. Polymeric or non-polymeric coatings, films, colloids, gels, etc. are contemplated. The formulation of the compositions will vary depending on the application. For example, when the composition is intended for coating a surface, e.g., a thin-film coating, paint layer, a film-forming polymer is often used in the formulation. If the composition is intended for use in sealing joints a combination of polymers useful as caulking agents may be selected.

Decontaminating coating, e.g., films, may be transparent, translucent, or opaque, depending on the intended use. For example, a window or lens might be coated with a transparent or translucent film, whereas an opaque film could be used to coat a wall or other surface for which the transmission of light is not required. The film may provided a protective barrier to the underlying surface. In some cases, the film may have an adhesive layer which bonds the film to the surface. Peel-off films are also envisioned, including multiple layer peel off films where layers may be peeled off to reveal a fresh film surface.

Examples of polymers that may be used in the compositions include, but are not limited to, poly(acrylic acid), poly(methacrylic acid), poly(methyl acrylate), poly(methyl methacrylate), polyimide, poly(amide imide), polyamide, polystyrene, soluble polyurethane, unsaturated polyester, poly(ether sulfone), poly(ether imide), poly(vinyl ester), polyurethane, silicone, and polyepoxide.

The decontaminating compositions may be produced using methods well known in the art. Typically, the methods involve combining an appropriate polymer, or combination of polymers, with an effective amount of a composite. In some case, the decontaminating compositions may be produced by adding an effective amount of a composite to an off-the-shelf product, such as, for example, a latex-based paint product. Vigorous mixing, such as by shaking, sonication, or vortexing, may facilitate dispersion of the composite in the polymer solutions. In some cases, the initial solubilization of the composite in water may aid its subsequent dispersion in the polymer solutions. Accordingly, in some cases, an aqueous solution comprising the composite may be mixed with a solution comprising an organic solvent and the polymer. Any organic solvent may be used, including any of the following non-limiting examples: toluene, diethyl ether, dichloromethane, chloroform, tetrahydrofuran, acetone, acrylamide, benzene, carbon disulfide, ethylene oxide, n-hexane, hydrogen sulfide, methanol, methyl mercaptan, methyl-N-butyl ketone, perchloroethene, styrene, methyl chloroform, trichloroethene, vinyl chloride, acetonitrile, dimethylformamide, dimethylsulfoxide, mesitylene, hexanes, decane, octane, nonane, diethylether, tetrahydrofuran, or xylene. However, depending on the polymer and/or mixing conditions, the use of solvents such as acetone, which are miscible with water, may avoid phase separation encountered by more hydrophobic solvents such as toluene and chloroform.

The decontaminating compositions may exist in a variety of forms. For example, the composition may be in the form of a fluid or solid. The decontaminating composition may be in the form of a film, e.g., a film having a thickness in a range of about 1 μm to about 2 μm, about 2 μm to about 5 μm, about 5 μm to about 10 μm, about 10 μm to about 20 μm, about 20 μm to about 50 μm, about 50 μm to about 100 μm, about 100 μm to about 200 μm, about 200 μm to about 500 μm, about 500 μm to about 1 mm, or about 1 mm to about 2 mm. The decontaminating composition may be in the form of a film having a thickness in a range of about 1 μm, about 2 μm, about 5 μm, about 10 μm, about 20 μm, about 50 μm, about 100 μm, about 200 μm, about 1 mm, about 2 mm, or more.

The decontaminating compositions may be latex paint formulations comprising a composite. Accordingly, the compositions may comprise one or more polymers suitable for use in a latex-based paint, binder(s), including, for example, acrylic resin, polyurethane resin, polyester resin, melamine resin, or epoxy resin binders, and/or any of a variety of pigments.

Any appropriate surface may be coated with the compositions disclosed herein. For example, the surface may be a plastic, a metal, a wood, a paper, a composite, a polymeric, a ceramic or a textile surface. Similarly, any of a variety of objects may be coated with the compositions, including, but not limited to, electronics equipment, e.g., radios, cell phones, GPS devices, communication equipment, computer equipment, displays, laptops; personal protective equipment, e.g., clothing, helmets, glasses, inflatable jackets, gas masks, flotation devices; military equipment, e.g., weapons, tanks, other vehicles; personnel carriers; cleaning equipment, e.g., mops, brooms, brushes, cloths, sponges; research equipment, e.g., tissue, cell, or virus culture equipment, fume hoods, culture hoods, labware, plasticware, glassware; and others, e.g., countertops, walls, ceilings, tiles, windows, personal care products, towels, clothing, mirrors, sporting equipment.

Methods of coating a surface with a composition are also provided herein. The methods typically involve depositing a film or layer of the composition onto the surface using any of a variety of art known techniques. Depending on the particular composition used and thickness of the coating desired, a surface may be coated with any of a variety of methods. For example, thin-films may be produced by chemical solution deposition, chemical vapor deposition or physical vapor deposition. In some cases, the surface may be spin-coated with the composition to achieve a thin-film coating. Thicker films may be produced by covering surface with the composition in liquid form and allowing the solvent to evaporate, thereby forming a solid coating. Solvent evaporation may be performed at ambient pressure or under vacuum.

If the composition is a paint, e.g., a latex-base paint, comprising a composite, the composition may be applied to the surface using a painting method appropriate for the particular paint formulation, e.g., brush-coat, roll-coat, spray-coat.

Methods for decontaminating a surface are also provided. For example, a surface which has been coated with a decontaminating composition may be decontaminated by contacting the coated surface with a peroxide, e.g., H₂O₂ and an acyl donor substrate, e.g., an acetate ester, e.g., propylene glycol diacetate (PGD), under conditions that produce sufficient levels of peracid, e.g., peracetic acid, to kill or eliminate any bacteria, yeasts, molds, fungi, and/or spores associated with the surface.

Typically, the decontaminating compositions comprise an effective amount of a composite. An effective amount of a composite is an amount of a composite sufficient to generate an activity level that is appropriate for a desired application. For example, an effective amount of a composite may be an amount of a composite sufficient to generate a perhydrolase activity capable of producing a peracid level that is suitable for killing bacteria, yeasts, molds, fungi, and/or spores in a desired application or under standard assay conditions. An effective amount of a composite may be an amount of a composite sufficient to generate a perhydrolase activity capable of producing a peracid level in a range of 0.005 mM to 0.01 mM, 0.01 mM to 0.05 mM, 0.05 mM to 0.1 mM, 0.1 mM to 0.5 mM, or 0.5 mM to 1 mM under standard assay conditions. An effective amount of a composite may be an amount of a composite sufficient to generate a perhydrolase activity capable of producing a peracid level of about 0.005 mM, about 0.01 mM, about 0.05 mM, about 0.1 mM, about 0.5 mM, about 1 mM, or more under standard assay conditions.

The concentration of a composite in a decontaminating composition may be in a range of about 0.001 μg/ml to about 0.01 μg/ml, about 0.01 μg/ml to about 0.1 μg/ml, or about 0.1 μg/ml to about 1.0 μg/ml, for example. The concentration of a composite in a decontaminating composition may be about 0.001 μg/ml, about 0.005 μg/ml, about 0.01 μg/ml, about 0.05 μg/ml, about 0.06 μg/ml, about 0.07 μg/ml, about 0.08 μg/ml, about 0.09 μg/ml, about 0.1 μg/ml, about 0.11 μg/ml, about 0.12 μg/ml, about 0.13 μg/ml, about 0.14 μg/ml, about 0.15 μg/ml, about 0.16 μg/ml, about 0.17 μg/ml, about 0.18 μg/ml, about 0.19 μg/ml, about 0.2 μg/ml, about 0.5 μg/ml, about 1.0 μg/ml, or more, for example.

Standard assay conditions for determining the perhydrolase activity of the decontaminating composition typically involve contacting the decontaminating composition with a peroxide, e.g., H₂O₂ and an acyl donor substrate, e.g., an acetate ester, e.g., propylene glycol diacetate (PGD) and measuring the level of peracetic acid produced over a predetermined time, e.g., about 20-30 minutes. If H₂O₂ is used in a standard assay, the concentration may be in a range of about 0.1 mM to 1 mM, about 1 mM to about 10 mM, about 10 mM to about 100 mM, or about 100 mM to about 500 mM. In some cases, the H₂O₂ concentration may be in a range of about 0.1 mM to about 400 mM. The H₂O₂ concentration may be about 0.1 mM, about 1 mM, about 10 mM, about 20 mM, about 50 mM, about 100 mM, about 200 mM, about 500 mM, or more. If PGD is used in a standard assay, the concentration may be in a range of about 1 mM to about 10 mM, about 10 mM to about 100 mM, or about 100 mM to about 500 mM. The PGD concentration may be about 1 mM, about 10 mM, about 20 mM, about 50 mM, about 100 mM, about 200 mM, about 300 mM, about 400 mM, about 500 mM, or more. If both H₂O₂ and PGD are used any appropriate combination of concentrations may be used. For example, the concentration of H₂O₂ may be about 100 mM and the concentration of PGD may be about 100 mM, the concentration of H₂O₂ may be about 100 mM and the concentration of PGD may be about 200 mM, the concentration of H₂O₂ may be about 200 mM and the concentration of PGD may be about 100 mM, or the concentration of H₂O₂ may be about 200 mM and the concentration of PGD may be about 200 mM.

### Decontaminating Kits

Decontaminating kits are also provided herein. The kits typically comprise a container housing a composite and instructions for using the composite to produce a decontaminating composition. The kits may also comprise at least one container housing a reagent for determining perhydrolase activity of the composite. The kits may also comprise at least one container housing a reagent for producing a decontaminating composition.

Decontaminating kits are also provided herein that comprise a container housing the decontaminating composition and instructions for coating a surface with the decontaminating composition. The kits may also comprise at least one container housing a reagent for determining perhydrolase activity of the composite of the decontaminating composition. The kits may also comprise at least one container housing a reagent for decontaminating the surface and instructions for decontaminating the surface using the reagent.

### EXAMPLES

### Introduction

Aspects of the examples involve covalent attachment of perhydrolases to carbon nanotubes and subsequent incorporation of the resulting conjugates into polymers (poly(methyl methacrylate) (PMMA) and poly(vinyl acetate) (PVAc)), and latex paint compositions. Carbon nanotubes were chosen as the nano-sized carriers (nanoparticles) for AcT because of their known ability to stabilize enzymes (20, 21). Moreover, structural and physical properties, such as high aspect ratios, low densities, and very high mechanical strength (22) make them excellent filling materials to reinforce polymers (23-25) and ceramics (26), in certain applications.

Significant effort has been made to address the generation of PAA from an aqueous environment through identification of perhydrolases with greater reactivity on H₂O₂ than on water as the acyl acceptor. In particular, a S54V variant of a perhydrolase (denoted as AcT) from Mycobacterium smegmatis is active on various acyl donor substrates and exhibits a perhydrolysis to hydrolysis ratio greater than 1. This results in perhydrolase activity 50-fold higher than that of the best lipase tested (18, 19). AcT is also stable in the presence of H₂O₂ and peracids.

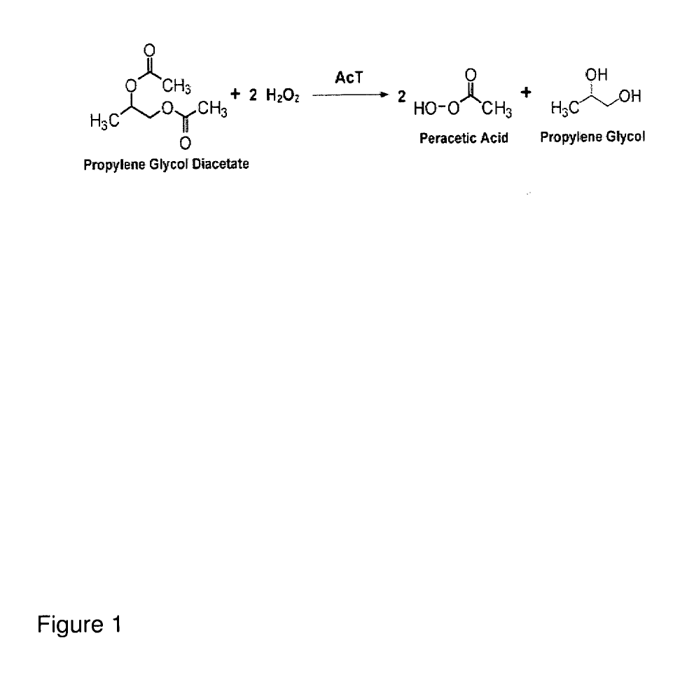

In aspects of the current disclosure, Applicants have exploited the interaction of AcT with multi-walled carbon nanotubes (MWNTs) to produce composites that allow efficient incorporation of the enzyme into polymeric coatings and paint. Enzyme-MWNT-material composites have been evaluated for their ability to generate PAA by perhydrolysis of propylene glycol diacetate (PGD) in the presence of H₂O₂ (FIG. 1). The incorporation of AcT-MWNT conjugates into polymers and paints is a step in the preparation of composites with enhanced strength and extended lifetime. These composites may be applied as decontaminating coatings on surfaces in hospitals, kitchens, and bathrooms, where effective killing of a variety of infectious organisms is critical.

### Materials and Methods

### Materials

Perhydrolase S54V (AcT) solution was provided by Genencor International, Inc. (Palo Alto, Calif.). MWNT (purity >95%, outer diameter 15±5 nm, length 5-20 μm) was purchased from NanoLab, Inc. (Newton, Mass.). Sulfuric acid (H₂SO₄, 95-98%), nitric acid (HNO₃, 68%-70%), and cover glass (circular, 25 mm) were purchased from Fisher Scientific (Hampton, N.H.), Propylene glycol diacetate (PGD), 2-(N┐morpholino)ethanesulfonic acid sodium salt (MES), hydrogen peroxide solution (30%), and uranyl acetate were purchased from Sigma (St. Louis; MO). 1-ethyl-3-[3-dimethylaminopropyl]carbodiimide hydrochloride (EDC) was purchased from Acros Organics (Morris Plains, N.J.). BCA protein assay kit, N-hydroxysuccinimide (NHS), and (2,2′-Azinobis [3-ethylbenzothiazoline-6-sulfonic acid] (ABTS) were purchased from Pierce (Rockford, Ill.). Isopore filter membrane (pore size 0.2 μm, type GTTP, polycarbonate) was purchased from Millipore (Billerica, Mass.). Amino-dPEG₁₂-acid was purchased from Quanta Biodesign (Powell, Ohio). Poly(methyl methacrylate) (PMMA, average Mw 996000) and poly(vinyl acetate) (PVAc, average Mw 500000)) were purchased from Aldrich (Milwaukee, Wis.). Latex enamel (gloss white, manufactured by Yenkin-Majestic Paint Corporation, Columbus, Ohio) was purchased from a local store.

### Functionalization of Carbon Nanotubes

Carboxylic acid groups were created on MWNTs by acid treatment. Typically, 100 mg MWNTs were added to an acid mixture containing 45 ml H₂SO₄ and 15 ml HNO₃ (H₂SO₄:HNO₃=3:1, v/v) and the suspension was sonicated at room temperature for 6 h in a VWR ultrasonic cleaner (model 50T, frequency from 38.5 to 40.5 kHz, average power of 45 W). The suspension of functionalized MWNTs was then diluted in 200 ml Milli-Q water and filtered through a 0.2 μm filter membrane. The nanotubes on the membrane were redispersed in 200 ml Milli-Q water by sonication and filtered again. This process was repeated at least six times to remove residual acids and any solubilized impurities. The functionalized MWNTs were dried under vacuum and stored at room temperature.

### Preparation of Act-MWNT Conjugates

AcT was covalently attached to functionalized MWNTs via a two-step process involving EDC/NHS activation followed by enzyme coupling (30). In a further example, amino-dPEG₁₂-acid was used as spacer between AcT and MWNT. Typically, 2 mg functionalized MWNTs were dispersed in 2 ml of MES buffer (50 mM, pH 4.7) containing 160 mM EDC and 80 mM NHS by brief sonication. After 15 min-shaking at 200 rpm at room temperature, the NHS activated MWNTs were filtered through 0.2 μm filter membrane and washed thoroughly with MES buffer. NHS-MWNTs were used immediately in the enzyme coupling reaction. For direct enzyme immobilization, 2 mg NHS-MWNTs were dispersed in 10 ml potassium phosphate buffer (50 mM, pH 7.1) containing 4 mg AcT and the enzyme coupling was allowed to proceed for 3 h at room temperature by shaking at 200 rpm. The AcT-MWNT conjugates were filtered and washed extensively with potassium phosphate buffer to remove free enzymes. The AcT┐dPEG-MWNT conjugates were prepared by first covalently attaching amino-dPEG₁₂-acid (used at 1 mg/ml in the reaction) to MWNTs and then attaching AcT to dPEG-MWNTs following the same two-step process as previously described.

### Preparation of Polymer and Paint Composites

Thin AcT-nanotube-polymer films were prepared by spin-coating (spin processor model: WS-400E-6NPP-LITE, Laurell Technologies Corporation, North Wales, Pa.) the conjugate-polymer solution at 4500 rpm for 50 sec on cover glass. The conjugate-polymer solution was prepared by mixing water suspension of AcT-dPEG-MWNT conjugates and acetone solution of PMMA (0.08 g/ml) or PVAc (0.1 g/ml) by vortexing. The thickness of each polymer film was measured using a profilometer (Dektak 8, Veeco Instruments Inc., Plainview, N.Y.). To prepare, thick films, the conjugate-polymer solutions (1 ml) were added in a glass vial (2.5 cm diameter) and the solvents were evaporated under vacuum. The AcT-nanotube-paint composites were prepared by adding water suspension of AcT-nanotube conjugates into latex (typically 0.2 ml) in a glass vial (2.5 cm diameter). The two components were mixed thoroughly using a pipette tip and the mixture was air-dried.

### Enzyme Loading

The concentration of AcT in solutions was measured using the standard BCA method. Briefly, the working reagent was prepared by mixing 50 parts of reagent A with 1 part of reagent B. AcT solution (50 μL) was added to the working reagent (1 ml) and the mixture was incubated at 37° C. for 30 min followed by measuring the absorbance at 562 nm on a UV-Vis spectrophotometer (Shimadzu UV-2401). Series dilution of AcT was performed to create the calibration curve. The amount of AcT attached on MWNTs was determined by subtracting the amount of enzyme washed out in the filtrates from the amount of AcT initially added. Alternatively, the AcT loading on nanotube was determined by elemental analysis (analyzed by Galbraith Laboratories Inc., Knoxyille, Tenn.).

### Activity Assays

The activity of AcT was determined by measuring the PAA generated from the reaction (55). The enzymatic reaction and PAA assay were performed following a standard protocol (provided by Genencor) with modifications. In a typical reaction, 10.6 μL, H₂O₂ stock solution (final concentration 100 mM) was added to a mixture of 0.8 ml PGD solution (final concentration 100 mM in potassium phosphate buffer, 50 mM, pH 7.1) and 0.2 ml AcT solution (2.0 μg/ml final concentration for free AcT or equivalent concentration of AcT for AcT-nanotube conjugates). The mixture was shaken at 200 rpm for 20 min at room temperature. PAA assay was conducted by diluting 25 μL of reaction solution 100-times in deionized water and subsequently mixing 25 μL of the diluted solution with 75 μL deionized water and 0.9 ml assay reagent (the assay reagent was prepared by mixing 5 ml potassium citrate buffer (125 mM, pH 5.0) with 50 μL ABTS water solution (100 mM) and 10 μL KI water solution (25 mM)). The mixture was then incubated at room temperature for 3 min and the absorbance at 420 nm was measured on a UV-Vis spectrophotometer. PAA concentration was calculated by [Peracetic Acid] (mM)=A420 nm×0.242×400 (400 is the dilution factor). The specific activity of AcT nanotube conjugates was calculated as the ratio of the normalized activity of the conjugates to that of the native AcT.

The activity of the composites (polymer films and paint) was measured by adding 0.8 ml POD solution (final concentration 100 mM), 0.2 ml buffer, and 10.6 μl H₂O₂ solution (final concentration 100 mM) into the container containing the polymer film or paint. After incubation at room temperature for 20 min, 25 μL of solution was withdrawn and PAA assay was conducted as described above.

Kinetics of AcT (free AcT and AcT-nanotube conjugates) was studied by measuring the initial reaction rates at different substrate concentrations. The concentration of hydrogen peroxide was varied from 0.1 mM to 428 mM while maintaining the PGD concentration at 200 mM.

### Dispersity Analysis

The dispersity of functionalized MWNT and AcT-nanotube conjugates in water was determined by centrifuging the corresponding water suspension (initial concentration 8 mg/ml for MWNT and 4 mg/ml for AcT-nanotube conjugates) at 3000 rpm for 5 min and then filtering 0.8 ml of the supernatant through a 0.2 μm membrane. After complete drying under vacuum, the amount of MWNT or AcT-nanotube conjugates on the membrane was measured and the dispersity was calculated based on the volume. Values obtained in this analysis did not reflect the saturation dispersity, which is actually the corresponding solubility.

### Sample Imaging

The morphology of AcT-MWNT conjugates was viewed by transmission electron microscopy (TEM) with a field emission gun at 120 kV (Phillips, CM-12). Typically, 10 μL, of the conjugate solution in water was dropped on Formvar carbon-coated grid (from Election Microscopy Sciences, Hatfield, Pa.) and then exposed to a 0.5% solution of uranyl acetate for ca 3 s. The samples were vacuum-dried overnight prior to TEM imaging.

### AcT Immobilization on Silica Nanoparticles

Silica nanoparticles (SiNP) were used for AcT enzyme immobilization. In one experiment, ca. 8 mg of silica nanoparticles (diameter of 15±5 nm, EKA Chemicals, Inc., Augusta, Ga.) were diluted in 1 ml dry ethanol and washed for at least 3 times. The samples were centrifuged at 10000 rpm for 5 minutes followed by re-dispersion in dry ethanol by sonication. In the second experiment, nanoparticles were functionalized with n-octadecyltrimethoxysilane (n-ODMS). Specifically, washed nanoparticles were sonicated in a solution of 1% (v/v) n-ODMS in dry ethanol for 2 hours. Subsequently, n-ODMS-functionalized silica nanoparticles were washed in dry ethanol 3 times followed by 3 washes in phosphate buffer (PB) by repeated centrifugation and sonication as described above.

To attach AcT, non-functionalized or n-ODMS functionalized nanoparticles were suspended in 0.4 mg/ml AcT in PB and incubated for 2 h at room temperature with shaking at 200 rpm. The resulting nanoparticle-protein conjugates were washed 6 times in PB by repeated centrifugation at 7000 rpm for 5 minutes and re-dispersed by pipetting. Supernatants from all of the washes were collected and the protein content was measured using the bicinchoninic acid (BCA) assay. SiNP-protein conjugates were used immediately or stored at 4° C. All reagents are purchased from Fisher, US unless otherwise mentioned.

In addition, for covalent immobilization silica nanoparticles derivatized with carboxyl groups were purchased from Life Science, Inc. AcT was attached using a similar protocol as for nanotube functionalization.

### Attenuated Total Reflection Fourier Transform Infrared Spectroscopy (ATR-FTIR)

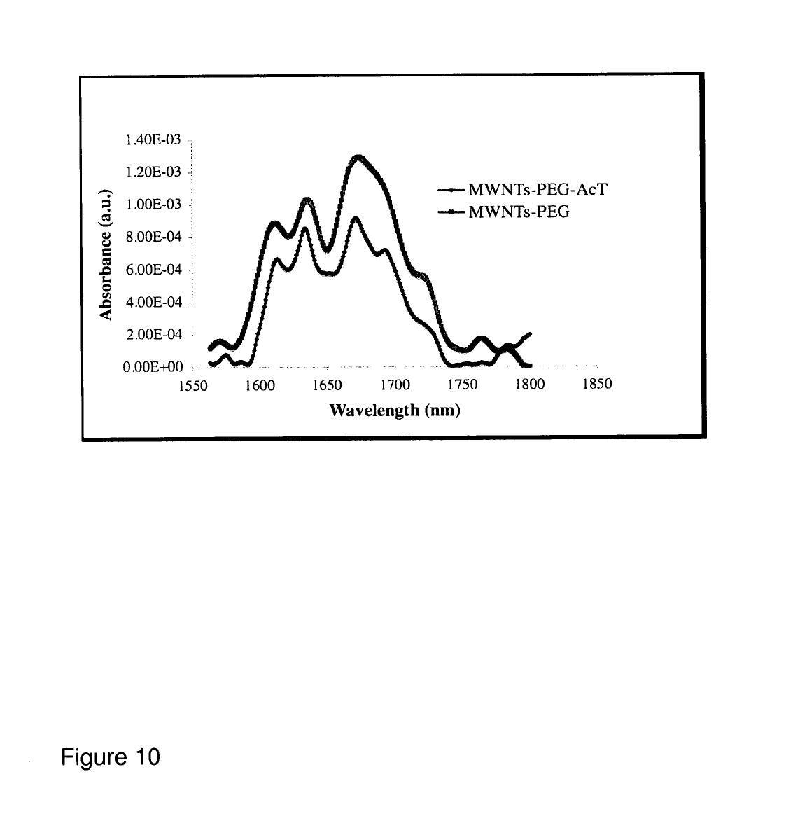

ATR-FTIR was used to determine the presence of amide bonds before and after the protein was chemically grafted on nanotubes (FIG. 10). A Nicolet Magna 550 Series II FTIR spectrometer (Madison, Wis.) with a horizontal attenuated total reflection (ATR) accessory (Spectra Tech Inc., Shellton, Conn.) was used to collect spectra of nanotube solutions (functionalized with PEG and AcT respectively). The ATR accessory has a trapezoidal germanium crystal (7.0×1.0 cm) with ends cut to 45° mounted onto a sample trough, generating 12 internal reflections. The spectrometer is equipped with a liquid nitrogen-cooled mercury cadmium telluride detector. To reduce the contributions of water vapor and carbon dioxide, the IR system was continuously purged with air from a FTIR purge gas generator (Model 74-45, Balston, Inc., Haverhill, Mass.) at 30 standard cubic feet per minute and supplemented with nitrogen gas from the vent of a liquid nitrogen tank. For obtaining the spectra and the corresponding background, approximately 500 μl of solution was carefully spread evenly over the germanium crystal for complete surface coverage. To prevent evaporation during spectra acquisition the crystal was covered with a parafilm and sealed using the accessory cover. For each spectrum, a 256 scan double-sided interferograms with Happ-Genzel apodization was collected at 2 cm-1 resolution in the range 1000-4000 cm⁻¹. The gain was set to 8 and an aperture of 40 was used. Amide I band, (1600-1700 cm⁻¹, centered at 1656 cm⁻¹) were recorded. Omnic software (v. 6.1a) from Nicolet (Madison, Wis.) was used to subtract the background pegylated nanotube surface, and water vapor contributions from the protein covalently attached to the nanotube surface. All the spectra were baseline corrected before each subtraction. After each experiment, the exposed surface of the germanium crystal was cleaned using a five-step process [55]: (a) rinsing with deionized (DI) water, (b) soaking in a 1% (w/w) sodium dodecyl sulfate (SDS) solution for 10 min, (c) rinsing thoroughly with DI water, (d) rinsing thoroughly with a 50% (w/w) aqueous ethanol solution and (e) drying with compressed air filtered through cotton to remove oils and particulates.

### Production and Analysis of AcT-Carbon Nanotube Conjugates

High activity and good solubility/dispersibility of enzyme-nanotube conjugates are important in certain applications to construct practically useful composites. Aggregation of carbon nanotubes in both aqueous and organic solvents due to surface-surface van der Waals interactions reduces available surface areas for biomolecule attachment and may also prevent their efficient dispersion in a polymer or paint composite (27, 28). Functionalized MWNTs that have been oxidized via acid treatment (29) yielding free carboxylic acid groups were used. The acid-functionalized MWNTs were soluble in water up to at least 5 mg/ml following brief sonication.

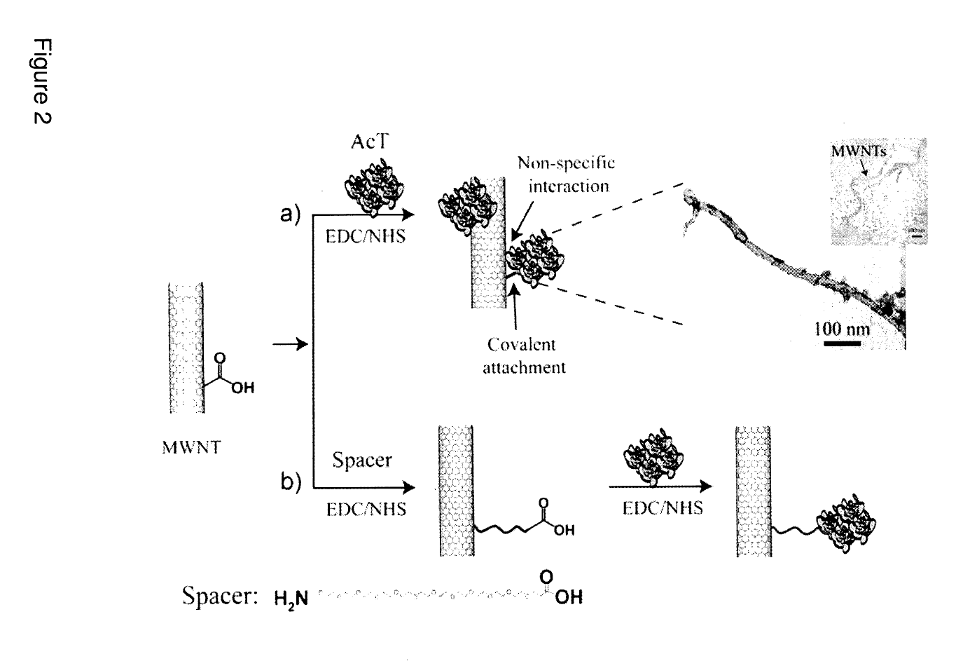

Covalent attachment of AcT to the water-soluble MWNTs was performed via EDC/NHS chemistry (30) (FIG. 2a) providing an AcT loading of 0.12 mg AcT per mg MWNTs, as determined by the BCA protein assay. The resulting AcT-MWNT conjugates were soluble to at least 2.5 mg/ml in aqueous buffer (50 mM potassium phosphate, pH 7.1). This solubility was deemed sufficient to provide uniform dispersion of the conjugates into polymeric and paint composites, which was expected to improve the activity of the composites by distributing the enzyme throughout the material (23, 31, 32).

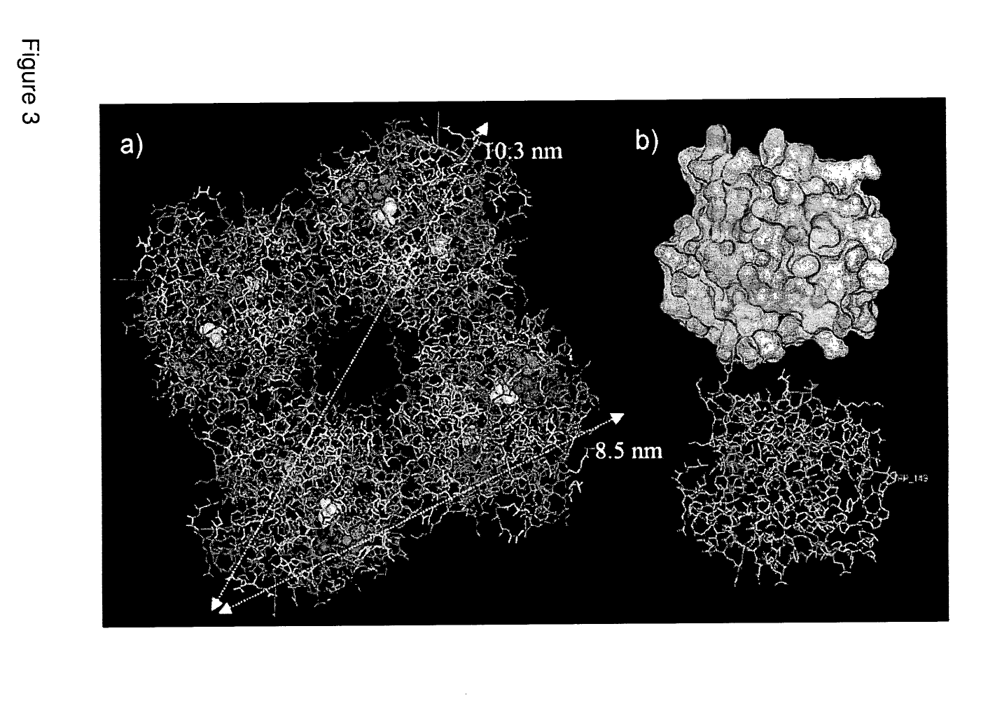

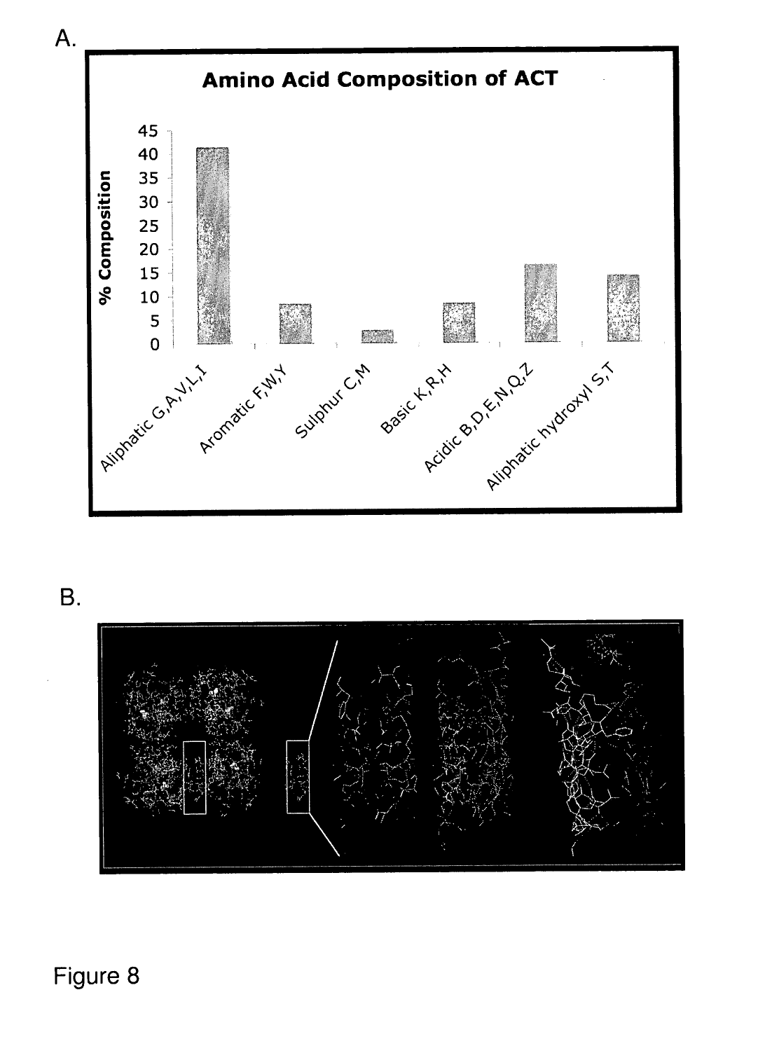

The AcT-MWNT conjugates retained about 7% of the native AcT activity. Changing the conditions for AcT attachment, such as varying the pH of the buffer and using different ratios of AcT/nanotube or EDC/NHS, was not observed to improve bound enzyme activity. The observed activity was substantially lower than that for other enzymes physically adsorbed onto carbon nanotubes. For example, carbon nanotube immobilized glucose oxidase (G0x) retained 68% of the free GOx activity (33) and lipase from Candida rugosa adsorbed on MWNT retained 97% of its biological activity (34). It has also been reported that soybean peroxidase (SBP) covalently attached to MWNTs retained 55% of the free SBP activity (35). AcT is a large molecule (an octamer, Mw=184 kDa) with dimensions of 72×72×60 Å (FIG. 3a) formed through tight association of pairs of dimers that may negatively impact the activity of the enzyme on supports, and hence on MWNT-based conjugates. Specifically, there are four insertions: residues 17-27; residues 59-69; residues 122-130; and residues 142-156 in the AcT structure, which form loops at the dimer interfaces and contribute to stabilization of the octameric structure. These loops are considered to enable formation of a hydrophobic channel that extends to the exterior of the octameric surface (FIG. 8b). The regions forming the hydrophobic channel lead to the active sites of the AcT being somewhat buried and thus having restricted substrate accessibility (18). Bioinformatic calculation (performed using ProtParam and images created on MOE, Chemical Computing Group Inc.) revealed that ˜60% of the amino acid residues that constitute the monomer are hydrophobic and the average hydropathicity of the monomer is 0.117 indicating a highly hydrophobic nature of the monomer (FIGS. 3b and 8a).

The large block-like structure and extensive hydrophobicity of AcT would presumably lead to substantial nonspecific hydrophobic interactions between the AcT surface and the non-functionalized hydrophobic regions of the MWNTs. These nonspecific hydrophobic interactions, together with covalent attachment, determine close packing of AcT molecules onto the MWNT surface (FIG. 2a, inset; as a comparison the bare acid-treated MWNTs are also shown). Consequently, the attached AcT molecules could have limited flexibility and their strong interaction with nanotube surface could also reduce the substrate accessibility to the active sites.

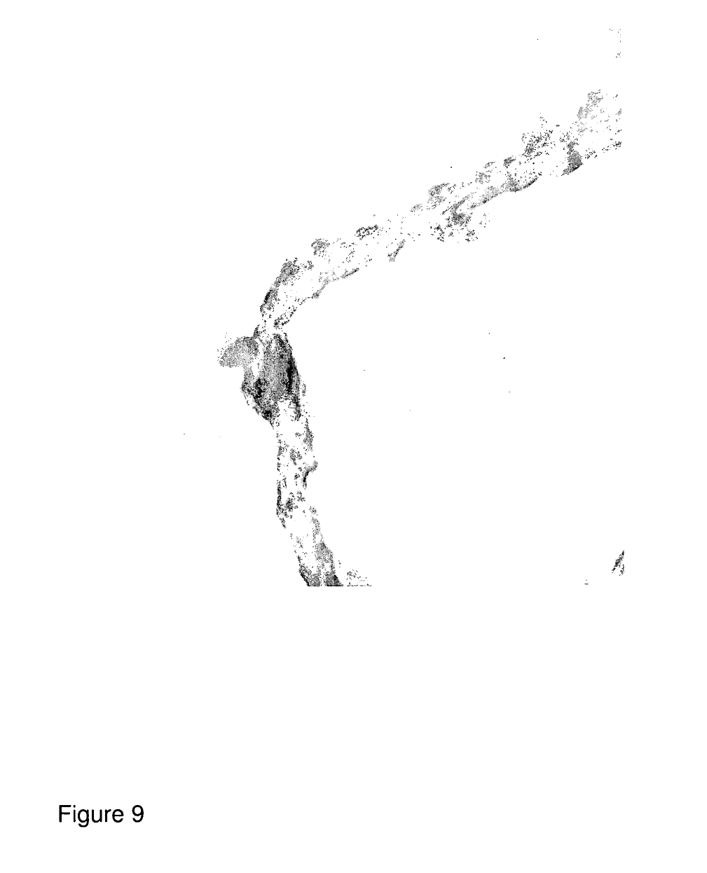

Enzyme flexibility can be altered by inserting a spacer between enzyme molecule and the attaching surface (36). Amphiphilic poly(ethylene glycol) (PEG) is a particularly effective linker, which is known to reduce nonspecific interactions (37), will not decrease the solubility of the carbon nanotubes (38, 39), and can enhance enzyme activity due to improved surface hydrophilicity (40). A bifunctional amino-dPEG₁₂ acid (dPEG, 4.7 nm in contour length) spacer was first covalently attached to the acid treated MWNTs and subsequently AcT was attached to the free end of the spacer both via EDC/NHS amide formation (FIGS. 2b and 9). Attenuated total reflection Fourier infrared spectroscopy (ATR-FTIR) was used to determine the presence of amide bonds before and after the protein was chemically grafted onto nanotubes (FIG. 10).

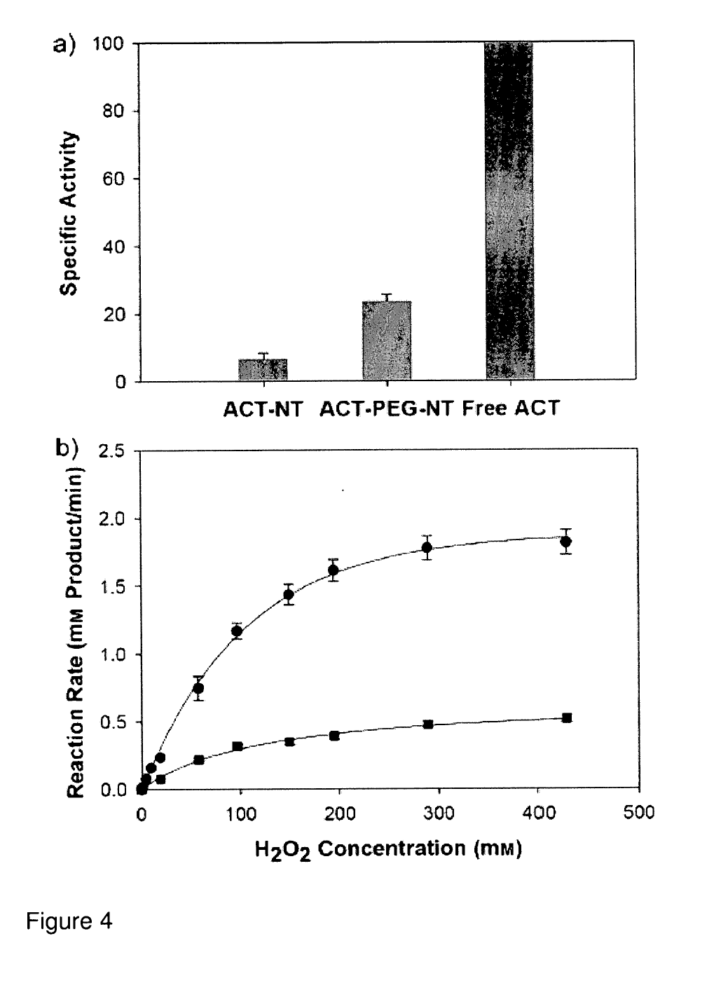

The dPEG spacer was effective in increasing the specific activity of the resulting AcT-dPEG-MWNT conjugates to 24% of that of free AcT (FIG. 4a). When 0.2 mg/ml nanotube and 0.4 mg/ml AcT were used in the coupling reaction, the resulting AcT-dPEG-MWNT conjugates had an enzyme loading of 0.06 mg AcT per mg of nanotube as determined by elemental analysis. As with the enzyme-MWNT conjugates without the dPEG linker, these conjugates were soluble up to 2.5 mg/ml in aqueous buffer. dPEG was used as the spacer in further preparations of AcT-nanotube conjugates.

Kinetic studies were performed for both free AcT and AcT-dPEG-MWNT conjugates by varying the concentration of H₂O₂ from 0.1 to 400 mM while maintaining the PGD concentration at 200 mM. Both the free AcT and the AcT-dPEG-MWNT conjugates followed Michaelis-Menten kinetics (FIG. 4b) with k_{cat} values of 4.6×10⁵ and 1.3×10⁵ min⁻¹ for free AcT and the AcT-dPEG-MWNT conjugates, respectively. Thus, the conjugate possessed ca. 28% of the intrinsic catalytic turnover as that of the free enzyme, indicating that the PEG linker markedly altered the reactivity of the enzyme when compared to the direct covalent attachment of the enzyme to the MWNTs. The Kₘ values were 115 and 123 mM for free AcT and AcT-dPEG-MWNT conjugates, respectively. Therefore, attachment of AcT onto functionalized MWNT via dPEG spacer did not significantly alter substrate-binding affinity. The good kinetic properties of the AcT-dPEG-MWNT conjugates led us to use this formulation for preparation of the polymer and paint composites.

### Production and Analysis of AcT-dPEG-MWNT-Composites

AcT-dPEG-MWNT conjugates retained high intrinsic catalytic activity and had high water-dispersity. The conjugates were incorporated into two industrially important polymers—poly(methyl methacrylate) (PMMA) and poly(vinyl acetate) (PVAc)—and into a latex paint. Incorporating bio catalysts into materials is desirable (41-47). In addition to direct crosslinking of biomolecules into the composite matrix, polymeric composites have also been prepared by interacting enzymes with a third component such as a different polymer (48), activated carbon (49), or carbon nanotubes (50, 51). With regard to the latter, it has been shown that both single- and multi-walled carbon nanotubes are able to stabilize enzymes in polymer composites (50, 51) eliminating the need to crosslink the enzymes within the network. The two-step process applied in this work also made it convenient to control the composite activity simply by varying the loading of AcT-dPEG-MWNT conjugates.

To prepare polymeric composites, water solutions of AcT-dPEG-MWNT conjugates were added to acetone solutions of PMMA or PVAc at a volume ratio of 1:15 (enzyme-based conjugates: polymer) and mixed by vortexing. The composites were then formed either by direct evaporation of the acetone and water in a glass vial or by spin-coating the solution onto a glass cover slide. The use of acetone as the solvent avoided phase separation, as may be encountered by more hydrophobic solvents, such as toluene and chloroform, while the initial solubilization of the conjugates in water aided their subsequent dispersion in the polymer solutions without sonication. In the case of paint composites, a 1:10 volume ratio of water solution of AcT-dPEG-MWNT conjugates to latex was used. Visually there was no phase separation for either the polymer or latex-based composites.

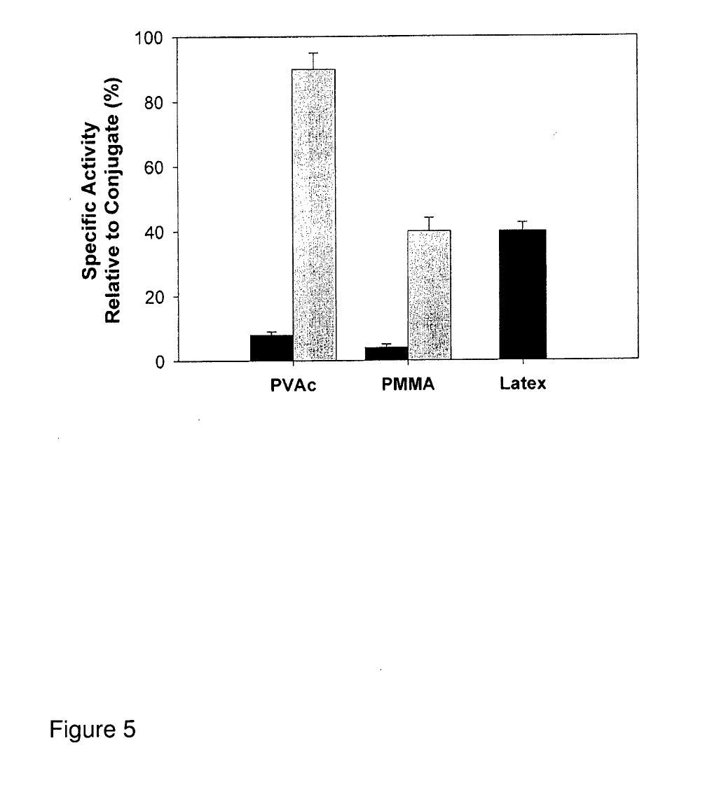

AcT activity in the PMMA and PVAc composites <10% of that of the AcT-dPEG-MWNT conjugates in aqueous solution (FIG. 5). These polymer films had a thickness of ˜200 μm, which could have limited the diffusion of PGD and H₂O₂ to the AcT, in some contexts. Indeed, estimation of the Thiele Modulus, ø (eq. 1), for H₂O₂ revealed a value of 230 indicating strong diffusional limitations.

$${φ}^{2} = {( \frac{h}{2} )}^{2} \frac{{v}_{max}}{{D}_{eff} {K}_{m}} \qquad ( 1 )$$

In eq. 1, h is the film thickness (200 μm) and νₘₐₓ is the maximal enzyme reaction rate (=k_{cat}×enzyme concentration). We used the value of k_{cat}=1.3×10⁵ min⁻¹ (as found in kinetic studies disclosed herein) and the enzyme concentration was obtained from the loading (40 μg (2.4 μg of AcT, 184 kDa enzyme molecular weight) in PMMA. The effective diffusivity (D_{eff}) was estimated to be 10⁻¹⁰ cm²/s. This value was based on water diffusion in a PMMA (molecular weight=834 kDa) film at a thickness of 200 μm (52).

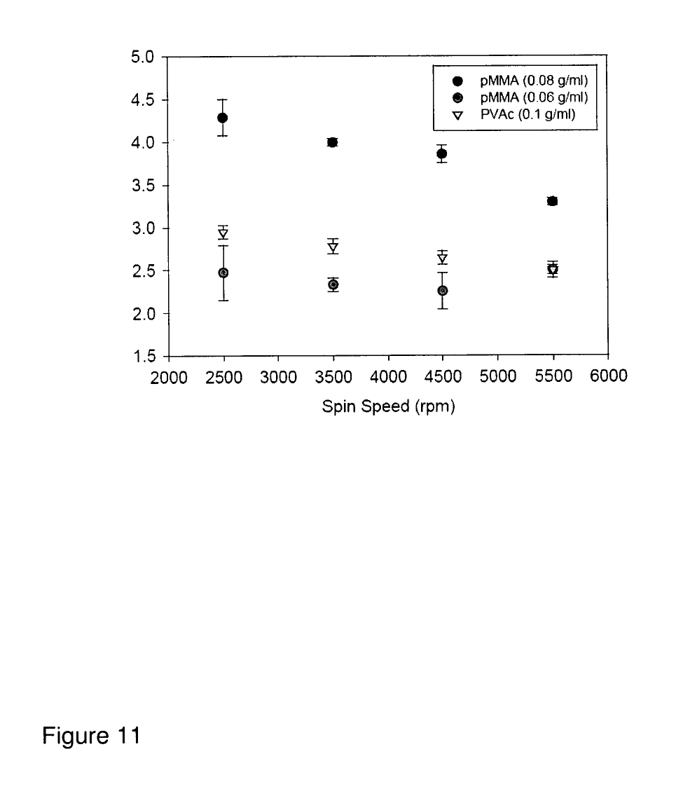

In addition to the diffusional limitations caused by the thick films, the large molecular size and hydrophobic character of AcT could lead to its extensive interaction with the surrounding hydrophobic PMMA and PVAc molecules, which could limit the accessibility of the AcT active sites to certain substrates. To address potential mass transfer issues in the polymeric composites, the film thickness was modified from 200 μm to ca. 2 μm by spin-coating the mixed solution of AcT-dPEG-MWNT conjugate and polymer (FIG. 11). The corresponding activity of the AcT-dPEG-MWNT-polymer films increased to ca. 40% and more than 90% of the conjugate activity for PMMA and PVAc, respectively (FIG. 5). The relatively hydrophilic nature of PVAc film may contribute to its higher activity when compared to the more hydrophobic and dense PMMA film. The latex paint composite exhibited ca. 40% of the conjugate activity even at a thickness of 400 μm.

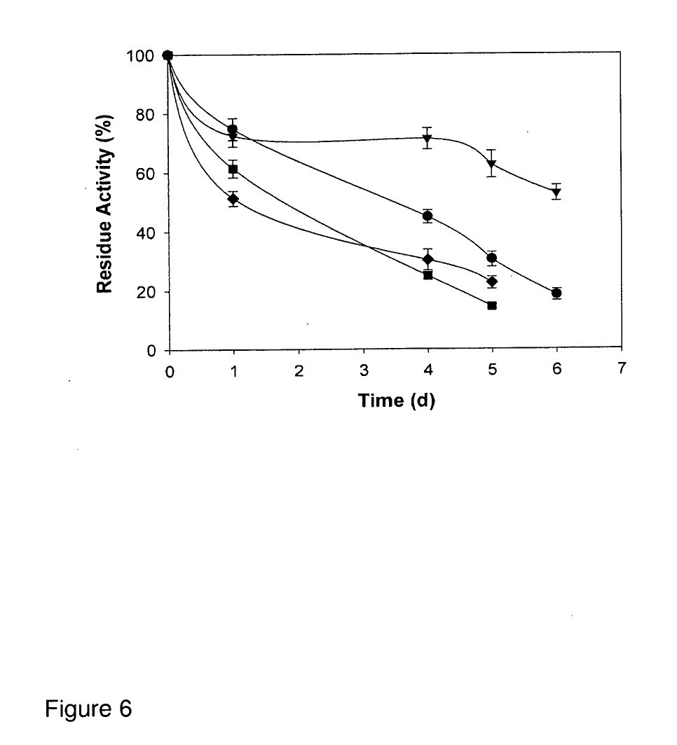

In addition to composite activity, composite stability and reusability were also examined. The spin-coated polymer films and the latex paint composites were stored under different conditions and their activity was measured every 24 h. The storage conditions were selected to mimic the environment that could be encountered in certain applications, and included storage in the dry state at room temperature and 50° C., and storage in the hydrated state (by immersing the composites in water) at room temperature and 50° C. The paint exhibited high stability when immersed in water at room temperature (FIG. 6). After a 6-day incubation and five reaction cycles, the paint retained >50% of its initial activity. When stored in dry state at room temperature the paint retained ˜20% of its original activity after 6-days and five reaction cycles. A similar trend was also observed at 50° C. with a more rapid loss in activity.

Without wishing to be bound by theory, the result whereby greater enzyme instability was observed in the dry state was considered as being due to residual PAA from the enzymatic reaction being retained in the dried AcT-containing paint. To test this hypothesis, the paints were incubated in the reaction solution for 1, 2, and 3 h after a typical 20-min reaction. The paints were then rinsed with water and air-dried. After 24 h, the paints retained 75%, 60%, and 30% of their original activity, respectively. This indicates that PAA is able to cause enzyme deactivation. In addition, to evaluate sample-handling effects (e.g., drying and washing) on the activity loss, the activity of the paint immersed in buffer was compared with that of the paint immersed in the reaction solution after five reaction cycles (1 day/cycle). The paint incubated in buffer exhibited no activity loss while the paint incubated in reaction solution showed a 60% activity loss. Without wishing to be bound by theory, it was reasoned that the residual PAA was able to diffuse out of the paint stored in buffer, and hence cause less deactivation of AcT. The PVAc and PMMA thin films showed similar stability trends as that of the paint under all testing conditions. When immersed in water, after 5 days and four reaction cycles the PVAc film retained 53% of its original activity, while after 4 days and three reaction cycles the PMMA film retained 62% of its original activity.

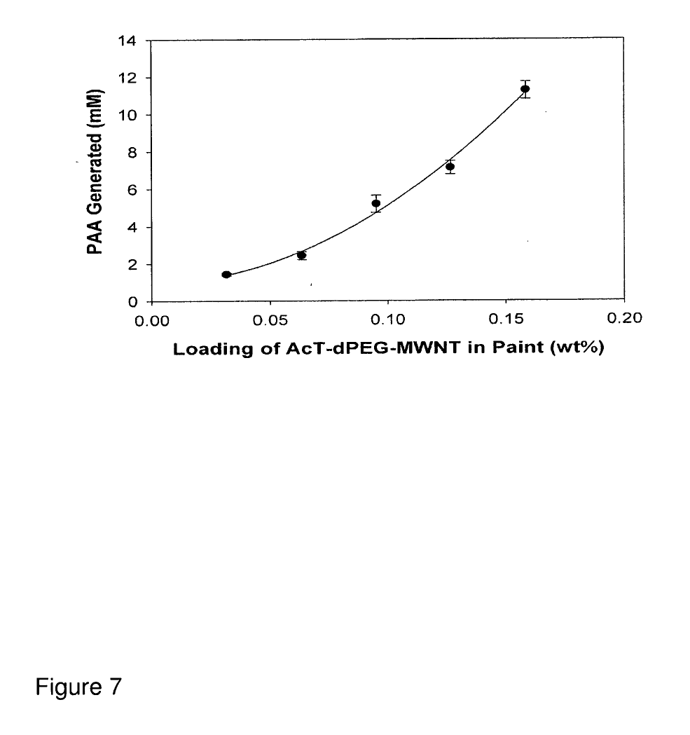

The ability of these composites to generate PAA is of interest in the development of decontaminating coatings. PVAc thin films (thickness ca. 2 μm, surface area of 5 cm²) containing a conjugate loading of 0.06 wt % (0.004 wt % of AcT) generated 0.2 mM PAA in 20 min; under the same conditions the thin PMMA film generated 0.05 mM of PAA at a conjugate loading of 0.08 wt % (0.005 wt % of AcT). Moreover, the paint composite (thickness ca. 400 μm) generated >11 mM PAA at a conjugate loading of 0.16% (0.01 wt % of AcT) (FIG. 7). Even though the thick films were diffusion limited, the larger amount of catalyst present (as compared to the spin-coated films) was able to yield higher levels of PAA. It has been reported that PAA is bactericidal at 0.13 mM, fungicidal at 0.39 mM, and sporicidal at 40 mM (53). In other tests, PAA was shown to effectively kill bacteria at concentration as low as 0.05 mM (54) and reduce spore CFUs 103.5-fold at a PAA concentration of 4 mM (6). Hence, the AcT-based paints would be expected to be highly microbicidal/sporicidal. Indeed, following 20 min incubation of the AcT-containing paint, the supernatant was capable of killing >99% of Bacillus cereus spores initially charged at 106 CFU/ml.

Highly water-soluble AcT-carbon nanotube conjugates were prepared and uniformly incorporated into polymer films and latex paint. The stability and reusability of the composites was evaluated. The capability of generating sufficiently high amount of potent PAA makes these composites useful as coating materials for disinfection in hospitals, food storages, and processing equipment against a wide range of pathogenic agents including bacteria, fungi, and spores.

### REFERENCES

1. Portner D M & Hoffman R K (1968) Appl. Environ. Microbiol. 16, 1782-1785.
2. Baldry M G C (1983) Journal of Applied Microbiology 54, 417-421
3. Small D, Chang W, Toghrol F, & Bentley W (2007) Applied Microbiology and Biotechnology 76, 1093-1105.
4. R. J. W. Lambert MDJEAS (1999) Journal of Applied Microbiology 87, 782-786.
5. Fatemi P & Frank J F (1999) Journal of Food Protection 62, 761-765.
6. Sagripanti J L & Bonifacino A (1996) Appl. Environ. Microbiol. 62, 545-551.
7. Kelsey J C, Mackinnon I H, & Maurer I M (1974) J Clin Pathol 27, 632-638.
8. Richard A. Ward R O (2003) Artificial Organs 27, 1029-1034.
9. Koivunen J & Heinonen-Tanski H (2005) Water Research 39, 4445-4453.
10. Zhao X, Zhang T, Zhou Y, & Liu D (2007) J. Mol. Catal. A: Chem. 271, 246-252.
11. Björkling F, Frykman H, Godtfredsen S E, & Kirk 0 (1992) Tedrahedron 48, 4587-4592.
12. Kirk O, Christensen M W, Damhus T, & Godtfredsen S E (1994) Biocatalysis and Biotransformation 11, 65-77.
13. Carboni-Oerlemans C, Dominguez de Maria P, Tuin B, Bargeman G, van der Meer A, & van Gemert R (2006) Journal of Biotechnology 126, 140-151.
14. Klaas MRg & Warwel S (1997) Journal of Molecular Catalysis A: Chemical 117, 311-319.
15. Rusch gen. Klaas M & Warvvel S (1999) Industrial Crops and Products 9, 125-132.
16. Ankudey E G, Olivo H F, & Peeples T L (2006) Green Chemistry 8, 923-926.
17. Rusch gen Klaas M, Steffens K, & Patett N (2002) Journal of Molecular Catalysis B: Enzymatic 19-20, 499-505.
18. Amin N S, Boston M G, Bott R R, Cervin M A, Concar E M, Gustwiller M E, Jones B E, Liebeton K, Miracle G S, OH. H, et al. (2006) in W02005056782 (A2), ed. WIPO (Genencor International, Inc, The Procter & Gamble Company, US), p. 523.
19. Mathews I, Soltis M, Saldajeno M, Ganshaw G, Sala R, Weyler W, Cervin M A, Whited G, & Bott R (2007) Biochemistry 46, 8969-8979.
20. Karajanagi S S, Vertegel A A, Kane R S, & Dordick J S (2004) Langmuir 20, 11594-11599.
21. Yim T-J, Liu J, Lu Y, Kane R S, & Dordick J S (2005) Journal of the American Chemical Society 127, 12200-12201.
22. Ajayan P M & Zhou O Z (2001) Topics in Applied Physics 80, 391-425.
23. B. Safadi RAEAG (2002) Journal of Applied Polymer Science 84, 2660-2669.
24. Lin Y, Zhou B, Fernando K A S, Liu P, Allard L F, & Sun Y-P (2003) Macromolecules 36, 7199-7204.
25. Qu L, Lin Y, Hill D E, Zhou B, Wang W, Sun X, Kitaygorodskiy A, Suarez M, Connell J W, Allard L F, et al. (2004) Macromolecules 37, 6055-6060.
26. Gao L, Jiang L, & Sun J (2006) J Electroceram. 17, 51-55.
27. Bahr J L, Mickelson E T, Bronikowski M J, Smalley R E, & Tour J M (2001) Chemical Communications, 193-194.
28. Bergin S D, Nicolosi V, Streich P V, Giordani S, Sun Z, Windle A H, Ryan P, Niraj N P P, Wang Z-T T, Carpenter L, et al. (2008) Advanced Materials (Weinheim, Germany) 20, 1876-1881.
29. Liu J, Rinzler A G, Dai H, Hafner J H, Bradley R K, Boul P J, Lu A, Iverson T, Shelimov K, Huffman C B, et al. (1998) Science (Washington, D.C.) 280, 1253-1256.
30. Jiang K, Schadler L S, Siegel R W, Zhang X, Zhang H, & Terrones M (2004) Journal of Materials Chemistry 14, 37-39.
31. Mitchell C A, Bahr J L, Arepalli S, Tour J M, & Krishnamoord R (2002) Macromolecules 35, 8825-8830.
32. Bhattacharyya S, Sinturel C, Salvetat J P, & Saboungi M L (2005) Applied Physics Letters 86, 113104.
33. Zhao X, Kim J, Jia H, & Wang P (2007) Preprints of Symposia—American Chemical Society, Division of Fuel Chemistry 52, 628-629.
34. Shah S, Solanki K, & Gupta M (2007) Chemistry Central Journal 1, 30.
35. Asuri P, Karajanagi S S, Sellitto E, Kim D Y, Kane R S, & Dordick J S (2006) Biotechnol Bioeng 95, 804-811.
36. Klaus Moeschel MNCSHB (2003) Biotechnology and Bioengineering 82, 190-199.
37. Lin Y, Allard L F, & Sun Y P (2004) J Phys. Chem. B 108, 3760-3764.
38. ShiraiFernando K A, Lin Y, & Sun Y P (2004) Langmuir 20, 4777-4778.
39. Chattopadhyay J, deJesusCortez F, Chalcraborty S, Slater N K H, & Billups W E (2006) Chem, Mater. 18, 5864-5868.
40. Loos K, Kennedy S B, Eidelman N, Tai Y, Zharnikov M, Amis E J, Ulman A, & Gross R A (2005) Langmuir 21, 5237-5241.
41. Wang P, Sergeeva M V, Lim L, & Dordick J S (1997) Nature Biotechnology 15, 789-793.
42. Novick S J & Dordick J S (1998) Chem. Mater. 10, 955-958.
43. Iqbal Gill A B (2000) Biotechnology and Bioengineering 70, 400-410.
44. Kim J, Kosto T J, II, Manimala J C, Nauman E B, & Dordick J S (2001) AlChE Journal 47, 240-244.
45. Vasileva N, Godjevargova T, Konsulov V, Simeonova A, & Turmanova S (2006) Journal of Applied Polymer Science 101, 4334-4340.
46. McDaniel C S, McDaniel J, Wales M E, & Wild J R (2006) Progress in Organic Coatings 55, 182-188.
47. Tong X, Trivedi A, Jia H, Zhang M, & Wang P (2008) Biotechnology Progress 24, 714-719.
48. Gill I, Pastor E, & Ballesteros A (1999) J Am. Chem. Soc. 121, 9487-9496.
49. Torras C, Tome V, Fierro V, Montane D, & Garcia-Valls R (2006) Journal of Membrane Science 273, 38-46.
50. Rege K, Raravikar N R, Kim D-Y, Schadler L S, Ajayan P M, & Dordick J S (2003) Nano Letters 3, 829-832.
51. Asuri P, Karajanagi S S, Kane R S, & Dordick J S (2007) Small 3, 50-53.
52. Ordaz I, Singh L, Ludovice P J, & Henderson C L (2006) Mater. Res. Soc. Symp. Proc. 899E, 0899-N0805-0805.0891-0896.
53. Greenspan F P & MacKeller D G (1951) Food Technot (Chicago, Ill., U.S.) 5, 95-97.
54. S. Stampi GDLFZ (2001) Journal of Applied Microbiology 91, 833-838,
55. Pinkernell U, Lüke H-J, & Karst U (1997) Analyst 122, 567-571.

All references described herein are incorporated by reference for the purposes described herein.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” “having,” “containing,” or “involving,” and variations thereof herein, is meant to encompass the items listed thereafter and equivalents thereof as well as additional items.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A composite comprising a nanomaterial associated with at least one perhydrolase.

2. The composite of claim 1, wherein the nanomaterial is a silica-based nanomaterial.

3. The composite of claim 1, wherein the nanomaterial is a carbon nanotube.

4. The composite of claim 3, wherein the carbon nanotube is a single-walled or multi-walled carbon nanotube.

5. (canceled)

6. The composite of claim 5, wherein the carbon nanotube has an outer diameter in a range of about 10 nm to about 20 nm.

7. The composite of claim 5, wherein the carbon nanotube has a length in a range of about 5 μm to about 2 μm.

8. (canceled)

9. The composite of claim 3, wherein the carbon nanotube is soluble in an aqueous solution at a concentration of up to 5 mg/ml.

10. The composite of claim 3, wherein the carbon nanotube is soluble in an aqueous solution at a concentration of up to 2.5 mg/ml.

11-12. (canceled)

13. The composite of claim 1, wherein the at least one perhydrolase is non-covalently associated with the nanomaterial.

14. (canceled)

15. The composite of claim 1, wherein the at least one perhydrolase is covalently associated to the nanomaterial.

16-18. (canceled)

19. The composite of claim 1, wherein the at least one perhydrolase is a member of the SGNH hydrolase protein family.

20. The composite of any claim 1, wherein the at least one perhydrolase comprises a GDSL motif.

21-22. (canceled)

23. The composite of claim 1, wherein the at least one perhydrolase comprises an amino acid sequence having at least 95% homology with the sequence set forth in SEQ ID NO: 1.

24. The composite of claim 1, wherein the at least one perhydrolase is isolated from Mycobacterium smegmatis.

25. The composite of claim 1, wherein the at least one perhydrolase has an amino acid sequence as set forth in SEQ ID NO: 3.

26. The composite of claim 1, wherein the at least one perhydrolase has a perhydrolysis to hydrolysis ratio of greater than one on an acyl donor substrate.

27. The composite of claim 26, wherein the acyl donor substrate is an acetate ester.

28. The composite of claim 27, wherein the acetate ester is propylene glycol diacetate (PGD).

29. The composite of claim 1, wherein the specific activity of the at least one perhydrolase on a PGD substrate is in a range of about 5% to about 25% of that of free perhydrolase.

30. The composite of claim 1, wherein the k_{cat} of the perhydrolase on a PGD substrate is in a range of about 0.1×10⁵ min⁻¹ to about 2.5×10⁵ min⁻¹.

31. The composite of claim 1, wherein the Kₘ of the perhydrolase on a PGD substrate is in a range of about 100 mM to about 150 mM.

32. The composite of claim 1, wherein the ratio of perhydrolase to nanomaterial is about 0.06 to 1.

33. The composite of claim 1, wherein the number of perhydrolase molecules per nanomaterial is in a range of 1 to 2000.

34-36. (canceled)

37. The composite of claim 1, wherein the perhydrolase catalyzes the perhydrolysis of an acetate ester to generate peracetic acid.

38. A composition comprising a polymer and the composite of claim 1.

39-41. (canceled)

42. The composition of claim 38, wherein the polymer is selected from the group consisting of: poly(acrylic acids), poly(methacrylic acids), poly(methyl acrylates), poly(methyl methacrylates), polyimides, poly(amide imides), polyamides, polystyrenes, soluble polyurethanes, unsaturated polyesters, poly(ether sulfones), poly(ether imides), poly(vinyl esters), polyurethanes, silicones, polyethers, and polyepoxides.

43-50. (canceled)

51. The composition of claim 38, wherein the specific activity of the perhydrolase of the composite in the composition on a PGD substrate is at least about 40% of the specific activity of the perhydrolase of a free composite on a PGD substrate.

52-53. (canceled)

54. A method of decontaminating a surface, the method comprising coating the surface with the composition of claim 38.

55-62. (canceled)

63. The method of claim 59, wherein the nanomaterial is a carbon nanotube and associating comprises:
activating the carbon nanotube, and
mixing a first solution comprising the activated carbon nanotube with a second solution comprising the perhydrolase under conditions that result in covalent attachment of the perhydrolase with the carbon nanotube.

64-80. (canceled)

81. A method of producing a decontaminating composition, the method comprising:
combining a polymer with an effective amount of a composite of claim 1.

82-103. (canceled)

104. A method of coating a surface with a decontaminating composition, the method comprising
providing a surface;
obtaining a decontaminating composition comprising a polymer and an effective amount of a composite of any one of claim 1; and
coating the surface with the decontaminating composition.

105-122. (canceled)

123. A composite comprising a nanomaterial associated with at least one haloperoxidase.

124-140. (canceled)

141. An object coated with any one of the compositions of claim 38.


SEQUENCE LISTING (3 sequences)

SEQ ID NO: 1 - protein, 216 aa, Mycobacterium smegmatis
MAKRILCFGDSLTWGWVPVEDGAPTERFAPDVRWTGVLAQQLGADFEVIEEGLSARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLL

SEQ ID NO: 2 - protein, 373 aa, Caldariomyces fumago
MFSKVLPFVGAVAALPHSVRQEPGSGIGYPYDNNTLPYVAPGPTDSRAPCPALNALANHGYIPHDGRAISRETLQNAFLNHMGIANSVIELALTNAFVVCEYVTGSDCGDSLVNLTLLAEPHAFEHDHSFSRKDYKQGVANSNDFIDNRNFDAETFQTSLDVVAGKTHFDYADMNEIRLQRESLSNELDFPGWFTESKPIQNVESGFIFALVSDFNLPDNDENPLVRIDWWKYWFTNESFPYHLGWHPPSPAREIEFVTSASSAVLAASVTSTPSSLPSGAIGPGAEAVPLSFASTMTPFLLATNAPYYAQDPTLGPNDKREAAPAATTSMAVFKNPYLEAIGTQDIKNQQAYVSSKAAAMASAMAANKARNL

SEQ ID NO: 3 - protein, 216 aa, Mycobacterium smegmatis
MAKRILCFGDSLTWGWVPVEDGAPTERFAPDVRWTGVLAQQLGADFEVIEEGLVARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLL